(19) United States
(12) Patent Application Publication
(10) Pub. No.: US 2024/0041558 A1
(43) Pub. Date: Feb. 8, 2024

(54) VIDEO-GUIDED PLACEMENT OF SURGICAL INSTRUMENTATION

(71) Applicant: The Johns Hopkins University, Baltimore, MD (US)

(72) Inventors: Jeffrey H. SIEWERDSEN, Baltimore, MD (US); Niral M. SHETH, Baltimore, MD (US); Prasad VAGDARGI, Baltimore, MD (US); Greg M. OSGOOD, Baltimore, MD (US); Wathudurage Tharindu DE SILVA, Baltimore, MD (US)

(73) Assignee: The Johns Hopkins University, Baltimore, MD (US)

(21) Appl. No.: 18/266,433

(22) PCT Filed: Dec. 9, 2021
(86) PCT No.: PCT/US2021/062702

§ 371 Date: Jun. 9, 2023

Related U.S. Application Data
(60) Provisional application No. 63/193,987, filed on May 27, 2021.
(60) Provisional application No. 63/123,909, filed on Dec. 10, 2020.

(51) Int. Cl. A61B 90/00; G06T 7/33; G06T 7/80

(52) U.S. Cl. CPC A61B 90/39; G06T 7/33; G06T 7/80; G06T 2207/10121; A61B 2090/3966; G06T 2207/10016; G06T 2207/10081; G06T 2207/30204; G06T 2207/10088; G06T 2207/30008

(57) ABSTRACT

A system for surgical navigation, including an instrument for a medical procedure attached to a camera and having a spatial position relative to the camera, an x-ray system to acquire x-ray images, and multiple fiducial markers detectable by both the camera and x-ray system, having a radio-opaque material arranged as at least one of a line and a point. A computer receives an optical image from the camera and an x-ray image from the x-ray system, identifies fiducial markers visible in both the optical image and x-ray image, determines for each fiducial marker a spatial position relative to the camera based on the optical image and relative to the x-ray system based on the x-ray image, and determines a spatial position for the instrument relative to the x-ray system based on at least the spatial positions relative to the camera and x-ray system.

### GOVERNMENT SUPPORT

This application claims priority to U.S. Provisional Application No. 63/123,909, filed Dec. 10, 2020, and to U.S. Provisional Application No. 63/193,987, filed May 27, 2021, which are incorporated herein by reference in their entirety.

This invention was made with government support under grant EB028330 awarded by the National Institutes of Health. The government has certain rights in this invention.

### BACKGROUND

### 1. Technical Field

Currently claimed embodiments of the invention relate to methods for intraoperative image-guided navigation of surgical instrumentation.

### 2. Discussion of Related Art

Orthopaedic trauma is a prominent socioeconomic burden in terms of lost quality of life and cost of surgery¹. In particular, fractures of the pelvic ring present a major challenge in orthopaedic trauma surgery, with an incidence rate of 37 out of 100,000 individuals/year (3-7% of all skeletal fractures)²⁻⁴ and considerably poor surgical outcomes with high complication rates (>30%) and mortality rates (8%)^{5,6}. Surgical treatment has commonly consisted of open or closed reduction followed by internal fixation, with percutaneous approaches under fluoroscopic guidance gaining prevalence in recent years due to shorter recovery times⁷⁻⁹.

Common surgical approach to fixation of pelvic fractures involves insertion of a guidewire (typically a Kirschner wire, K-wire) along bone corridors in the pubis, ischium, and/or ilium, followed by insertion of a cannulated screw along the K-wire trajectory (after which the guidewire is removed)¹⁰. The procedure is commonly guided by x-ray fluoroscopy on a mobile C-arm, where intermittent exposures are acquired concurrently with placement of the guidewire for assessment of device position relative to surrounding anatomy—namely conformance within bone corridors¹¹.

Surgeons cognitively, qualitatively estimate the 3D position of the K-wire within the pelvis from multiple projection views (e.g., inlet, outlet, at other oblique views). However, due to the challenge of 3D reckoning within the complex morphology of the pelvis, accurate K-wire placement often requires “fluoro-hunting” and multiple trial and error attempts, even for experienced surgeons. It is not uncommon for the guidewire to be completely withdrawn if the K-wire trajectory appears in danger of breaching the bone corridor and reinserted along a new trajectory, leading to extended procedure time and fluoroscopic exposure (often exceeding 120 seconds of fluoroscopy time and hundreds of radiographic views¹⁰). The ability to accurately place K-wires under fluoroscopic guidance requires long learning curves to achieve sufficient quality in device placement.

Surgical navigation has emerged as a potential solution for guidance of device placement and reduction of radiation dose. Current state-of-the-art navigation systems include the Medtronic StealthStation and Stryker Navigation System II, which use optical (infrared) tracking of rigid markers to provide virtual visualization of the location of surgical instruments with respect to preoperative or intraoperative CT, cone-beam CT (CBCT), or MRI. Navigation based on such trackers is fairly common in brain and spine surgery^{12,13}, where its use has demonstrated improved surgical precision. However, orthopaedic trauma surgery has not seen widespread adoption of these systems, primarily due to factors of cost, the requirement for external trackers with line of sight, and workflow bottlenecks associated with setup calibration/registration. With such a fast-paced environment and steep workflow requirements, fluoroscopic guidance remains the mainstay for surgical guidance in orthopaedic trauma, with the standard of care largely unchanged for decades.

One solution, specifically targeting procedures in orthopaedic trauma surgery, disclosed an invention describing the attachment of a calibrated, tracked marker to a surgical drill (U.S. Pat. No. 6,887,247, CAS drill guide and drill tracking system). However, the invention still required the maintenance of line-of-sight to an external infrared camera and suffers from the same limitations of mainstream navigation systems

As noted above, current state-of-the-art systems typically use optical (infrared) tracking of rigid markers to provide virtual visualization of the location of surgical instruments with respect to preoperative or intraoperative CT, cone-beam CT (CBCT), or MRI. To provide such visualizations, a registration between the surgical instrument (via the tracking system) and 3D intraoperative and/or preoperative images must be performed,

Typical registration procedures involve placing markers on the patient prior to 3D image acquisition, requiring the markers to remain in place until the start of the surgical procedure. At the start of the procedure, the marker positions on the patient and within the 3D image are matched to obtain the registration relating the surgical instrument to the patient. A common drawback in such navigation approaches is that any perturbations in the position of markers that occur between the initial preoperative setup and the time of procedure cannot be tracked.

Since any variation in marker location may adversely affect registration accuracy, prior solutions have sought to mitigate perturbations by invasively attaching markers to rigid structures within the patient, further adding to the cost, time, and complexity of the procedure. Other approaches have looked towards marker-less and surface-matching based registration methods. In marker-less registration, corresponding anatomical landmarks (e.g. distinct bony surfaces) between prior image data and the patient are manually identified by the surgeon. While such an approach avoids the use of invasive markers, it is subject to inconsistences between data collected during planning and the time of surgery (e.g. skin-surface deformations).

In conventional surface-matching approaches, a mapped surface of the patient is created by tracing a tracked pointer along the patient skin surface. The resulting surface is then registered to a corresponding surface segmented from image data. Such methods are subject to the same errors as the conventional marker-less approach, often require time-consuming setup and multiple, manual re-registration steps, further adding to the time and complexity of the procedure.

Surgical navigation solutions that are potentially better suited to the demanding workflow of orthopaedic trauma surgery have been reported in prior art. Some have mounted a camera on the C-arm to provide augmented fluoroscopic guidance and tool tracking.¹⁴⁻¹⁶ Others have aimed to improve accuracy and reduce line-of-sight obstruction—e.g., mounting a surgical tracker to the C-arm¹⁷—a solution that also provides stereoscopic video for augmented views of the surgical scene and/or fluoroscopy. Others incorporated the tracking system into the operating table—as in Yoo et al.¹⁸, who incorporated an open frame electromagnetic field generator into the operating table to provide real-time tracking while maintaining compatibility with fluoroscopy (i.e., the PA view shooting through the open frame). Still others have sought to mount tracking equipment on the instrument itself¹⁹⁻²¹.

Prior art in ultrasound needle-based interventions have disclosed the mounting of a video camera onto an ultrasound probe as a means to realize targeted needle guidance (U.S. Pat. No. 14,092,755, Ultrasound system with stereo image guidance or tracking; U.S. Pat. No. 14,689,849, System and method for fused image-based navigation with late marker placement).

In Magaraggia et al.¹⁹, a video-based guidance framework was described for real-time guidance of the surgical drill tip in distal radial fracture surgery. The framework took advantage of implant-specific drill sleeves by augmenting them with binary markers, allowing video-based tracking by a camera mounted on the surgical drill. The study reported improvements in screw positioning accuracy compared to conventional fluoroscopic guidance and compatibility with clinical workflow, recognizing that the approach is limited to surgeries that routinely employ drill sleeves.

Prior solutions that go beyond the conventional approach for fiducial registration have been reported. One such solution discloses the invention of a flexible tracking article that consists of a substrate containing multiple point-based features that can be detected by an external tracking system (viz. active LEDs detected by an external infrared camera). The point-based features are used to create a surface model of the patient that can then be registered to surfaces extracted from image data (e.g. preoperative CT or MRI) allowing automatic registration and tracking of surgical devices without the need for invasive markers. (U.S. Pat. No. 9,901,407, Computer-implemented technique for determining a coordinate transformation for surgical navigation; U.S. Pat. No. 7,869,861, Flexible tracking article and method of using the same).

Prior art has also used “multimodal” markers visible to both an external tracking modality and an x-ray imaging system to establish navigation in surgical procedures that routinely use fluoroscopic. imaging (e.g., orthopaedic-trauma surgery). In Hamming et al.²² and Dang et al.²³, multimodal markers comprised of an infrared reflective sphere enclosing a radio-opaque tungsten sphere were described. The works reported on various arrangements of these multimodal markers (e.g. predefined, known 3D configurations as well as free-form 3D configurations) as well as the corresponding methods used to automatically solve the registration between a stereoscopic infrared tracking system and intraoperative CBCT images. In Andress et al.²⁴, a multimodal marker (optical and radio-opaque) with features based on the well-known ARToolKit was used to co-register an augmented reality, head mounted display with fluoroscopy images.

### SUMMARY

In some embodiments, a system for surgical navigation, including an instrument configured for a medical procedure on a patient, a camera attached to the instrument, wherein the instrument has a spatial position relative to the camera, an x-ray system configured to acquire x-ray images of the patient during the medical procedure, and multiple fiducial markers positioned on the surface of the patient during the medical procedure, the fiducial markers being detectable by both the camera and the x-ray system, the fiducial markers comprising a radio-opaque material arranged as at least one of a line and a point. The system also includes a computer configured to receive an optical image acquired by the camera, receive an x-ray image acquired by the x-ray system, identify a subset of the fiducial markers that are visible in the optical image and are also visible in the x-ray image, determine, based on the optical image, a spatial position relative to the camera for each fiducial marker in the subset of fiducial markers, determine, based on the x-ray image, a spatial position relative to the x-ray system for each fiducial marker in the subset of fiducial markers, and determine, based on at least the spatial positions of the subset of fiducial markers relative to the camera and the spatial positions of the subset of fiducial markers relative to the x-ray system, a spatial position for the instrument relative to the x-ray system.

In some embodiments, a method for surgical navigation, including receiving an optical image acquired by a camera, the camera attached to an instrument configured for a medical procedure on a patient, the instrument having a spatial position relative to the camera. The method includes receiving an x-ray image acquired by an x-ray system configured to acquire x-ray images of the patient during the medical procedure. For multiple Uncial markers positioned on the surface of the patient during the medical procedure and detectable by both the camera and the x-ray system, the method includes identifying a subset of the fiducial markers that are visible in the optical image and are also visible in the x-ray image, the fiducial markers comprising a radio-opaque material arranged as at least one of a line and a point. The method includes determining, based on the optical image, a spatial position relative to the camera for each fiducial marker in the subset of fiducial markers, determining, based on the x-ray image, a spatial position relative to the x-ray system for each fiducial marker in the subset of fiducial markers, and determining, based on at least the spatial positions of the subset of fiducial markers relative to the camera and the spatial positions of the subset of fiducial markers relative to the x-ray system, a spatial position for the instrument relative to the x-ray system.

In some embodiments, a system for surgical navigation, including an instrument configured for a medical procedure on a patient, a camera attached to the instrument, the instrument having a spatial position relative to the camera, an x-ray system configured to acquire x-ray images of the patient during the medical procedure, and multiple fiducial markers positioned on the surface of the patient during the medical procedure, the fiducial markers being detectable by both the camera and the x-ray system. The system includes a computer configured to receive a two-dimensional (2D) optical image acquired by the camera, receive a 2D x-ray image acquired by the x-ray system, identify a subset of the fiducial markers that are visible in the optical image and are also visible in the x-ray image, determine, based on the 2D optical image, a spatial position relative to the camera for each fiducial marker in the subset of fiducial markers, determine, based on the 2D x-ray image, a spatial position relative to the x-ray system for each fiducial marker in the subset of fiducial markers, and determine, based on at least the spatial positions of the subset of fiducial markers relative to the camera and the spatial positions of the subset of fiducial markers relative to the x-ray system, a spatial position for the instrument relative to the x-ray system.

In some embodiments, a method for surgical navigation, including receiving a two-dimensional (2D) optical image acquired by a camera, the camera being attached to an instrument configured for a medical procedure on a patient, the instrument having a spatial position relative to the camera. The method includes receiving a 2D x-ray image acquired by an x-ray system configured to acquire x-ray images of the patient during the medical procedure. For a plurality of fiducial markers positioned on the surface of the patient during the medical procedure and detectable by both the camera and the x-ray system, the method includes identifying a subset of the fiducial markers that are visible in the optical image and are also visible in the x-ray image, determining, based on the 2D optical image, a spatial position relative to the camera for each fiducial marker in the subset of fiducial markers, determining, based on the 2D x-ray image, a spatial position relative to the x-ray system for each fiducial marker in the subset of fiducial markers, and determining, based on at least the spatial positions of the subset of fiducial markers relative to the camera and the spatial positions of the subset of fiducial markers relative to the x-ray system, a spatial position for the instrument relative to the x-ray system.

### BRIEF DESCRIPTION OF THE DRAWINGS

Further objectives and advantages will become apparent from a consideration of the description, drawings, and examples.

FIG. 1A illustrates a video-on-drill system of some embodiments.

FIG. 1B illustrates 3D-2D registration of the drill axis in fluoroscopy using the system of FIG. 1A.

FIC. 1C illustrates a 3D-printed drill mount to rigidly hold the video camera in the system of FIG. 1A.

FIG. 1D illustrates a video scene of markers placed about the drill entry point using the system of FIG. 1A.

FIG. 1E illustrates the same markers as in FIG. 1D, as seen in C-arm x-ray fluoroscopy.

FIGS. 2A-2D illustrates a workflow for a drill guidance system of some embodiments.

FIG. 3A illustrates the drill camera 3D coordinate frame, the C-arm 3D coordinate frame, and the preoperative CT 3D coordinate frame.

FIG. 3B illustrates a schematic of multimodal marker features for some embodiments visible in both video and fluoroscopy.

FIG. 4 illustrates a flowchart of some embodiments for the 3D-2D registration pipeline of intraoperative fluoroscopy to the markers and to preoperative CT.

FIG. 5A illustrates an exemplary experimental setup for evaluating the performance of the video-guided drill system of some embodiments.

FIG. 5B illustrates an example of the drill camera and markers in some embodiments.

FIGS. 6A and 6B illustrate errors associated with 3D marker localization from video images.

FIGS. 7A and 7B illustrate errors associated with 3D marker localization from fluoroscopic images.

FIGS. 8A and 8B illustrate the 3D-2D registration accuracy of preoperative CT to the C-arm coordinate frame.

FIGS. 9A and 9B illustrate registration performance for end-to-end drill axis localization in the preoperative CT coordinate frame.

FIG. 10A illustrates an embodiment of augmented fluoroscopy guidance.

FIG. 10B illustrates an embodiment of augmented CT guidance.

FIGS. 11A-11C illustrate conformance analysis and visualization of drill axis trajectories within different bone corridors.

FIGS. 12A and 12B illustrate an embodiment of an experimental setup of the video-on-drill system for pre-clinical evaluation.

FIGS. 13A and 13B illustrate drill axis registration error and conformance of drill axis trajectories.

FIGS. 14A-14D illustrate visualization and guidance in Video-Fluoro and Video-CT modes across multiple trajectories.

FIG. 15 schematically illustrates an image-guided navigation system of some embodiments using multimodal fiducials to co-register camera and x-ray scenes.

FIGS. 16A-16D illustrate embodiments of paired-point multimodal markers.

FIG. 17 shows a flowchart of the 2D-3D registration process for some embodiments using paired-point markers.

FIGS. 18A and 18B illustrate embodiments of paired-point marker arrangements that are compatible with clinical workflows.

FIG. 19 illustrates an embodiment of a paired-point based marker arrangement upon a patient surface.

FIGS. 20A-20D FIG. 20 illustrate embodiments of point-cloud multimodal markers.

FIG. 21 shows a flowchart of the 2D-3D registration process for some embodiments using point-cloud markers.

FIGS. 22A and 22B illustrate embodiments of point-cloud marker arrangements that are compatible with clinical workflows.

FIG. 23 illustrates an embodiment of a point-cloud based marker arrangement upon a patient surface.

FIG. 24 shows a flowchart for 3D-2D registration of multimodal markers in some embodiments.

FIGS. 25A-25D illustrate an embodiment of a video-guided drill guide, and provide a comparison to a video-guided drill embodiment.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed, and other methods developed, without departing from the broad concepts of the current invention. All references cited anywhere in this specification, including the Background and Detailed Description sections, are incorporated by reference as if each had been individually incorporated.

Some embodiments of the invention provide a video-based guidance system that is suitable to the workflow of fluoroscopy-guided orthopaedic trauma procedures, biopsy procedures, and other medical interventional procedures. Some embodiments of the system are applicable to surgical drills, biopsy needles, and other instruments that require real-time navigation.

Some embodiments of the invention provide a surgical guidance system that assists in the placement of surgical instruments. In some embodiments, the system comprises a video camera (or other tracker modalities including but riot limited to infrared and electromagnetic sensors) attached to a surgical drill (or other instrument); an x-ray imaging system (either plane radiography or fluoroscopy); and an arrangement of multimodal fiducial markers visible to both the attached tracking modality (e.g. video, infrared) and the x-ray system (radio-opaque). The underlying geometric relationships between the surgical instrument and x-ray imaging system are solved enabling the registration and overlay of instrument position onto x-ray images and/or preoperative 3D images (e.g. CT, MRI). Some embodiments improve the accuracy and safety of instrument placement, and decrease both x-ray imaging dose and procedure time in image-guided interventions.

In some embodiments, the instrument position is determined by determining the spatial position of the fiducial markers in two coordinate frames. From the optical images and known calibration parameters of the camera (mounted on the drill), the markers are localized with respect to the camera. From the x-ray projection images and a known geometric calibration of the x-ray imaging system (e.g. C-arm), the markers are also localized in the C-arm/lab frame. The markers identified in both coordinate frames (e.g., at least three) are then used to solve a registration between the two coordinate frames. This solved registration represents the camera's pose in the C-arm/lab frame (i.e., the camera's known position in the lab frame). With this registration (which is solved in real-time), the calibrated drill axis is then shown in both CT images and radiographic images (via projection).

In some embodiments, a miniature camera is mounted on a surgical drill to provide real-time visualization of the drill trajectory in fluoroscopy and/or CT. The relationship between camera images and intraoperative fluoroscopy is established via multimodality (optical and radio-opaque) fiducial markers that are placed about the surgical field at the time of surgery. Notably (and likely essential to realistic workflow), in some embodiments the markers do not need to appear in preoperative CT or MRI. The solution of some embodiments couples 3D-2D registration algorithms with vision-based tracking to register and track surgical instrumentation without additional equipment in the operating room (OR). The proposed solution also has the potential to reduce radiation dose in the OR by reducing “fluoro-hunting,” and registration can be performed in some embodiments with commonly acquired (e.g., inlet and outlet) views. In principle, the system could reduce the number of views required for K-wire placement from hundreds¹⁰ to as few as two projections—one for initial registration and one for visual confirmation of device placement.

In some embodiments, in place of an external tracker system, a video camera is attached to a surgical drill that holds the instrument. Compared to the drill-mounted video system described in Magaraggia et al.,¹⁹ some embodiments use multimodal markers (rather than custom drill sleeves) to register video images to fluoroscopy. As such, the underlying registration algorithms are different.

Some embodiments use automatic 3D-2D registration to register the tracked instrumentation (e.g., surgical drills, biopsy needles, etc.) to a preoperative CT (or intraoperative CBCT) enabling 3D navigation. Prior art video-based systems require that the video markers be placed on the patient in the preoperative CT.²⁵⁻²⁹ The need to place markers in preoperative imaging presents a workflow barrier that is not likely to be compatible with emergent trauma scenarios—where the preoperative image is acquired quickly for diagnostic purposes (e.g., rule-out hemorrhage or other conditions as well as detection and characterization of the fracture).

Most prior art systems also require the markers to remain unperturbed from the moment they are imaged through the duration of the case. Instead, some embodiments of the proposed system allow the markers to be placed during the procedure (e.g., after the patient is draped, immediately prior to K-wire insertion), and perturbations of the marker arrangement is accommodated by updating the registration with as little as one fluoroscopic view.

Some embodiments provide multimodal fiducial markers and corresponding automatic registration algorithms that overcome limitations of current state-of-the-art navigation methods. In some embodiments, the markers contain both optical (vision-based) and radio-opaque point-based features with known geometric correspondence. Notably (and likely essential to realistic workflow), in some embodiments the markers do not need to appear in preoperative imaging and are instead placed about the surgical field and registered at the time of surgery. Some embodiments utilize multimodal marker arrangements that are potentially compatible with existing clinical workflows.

Some embodiments describe “stray” multimodal (optical and radio-opaque) markers with potential arrangements of the “stray” markers for feasible usage in the clinic. Unlike conventional markers, some embodiments are robust to perturbations in marker position due to skin/surface deformation since the registration between the video scene and radiographic image updates with each x-ray shot. Some embodiments of the invention are compatible with preoperative images acquired in the diagnostic work-up prior to surgery day, obviating the need to acquire a preoperative CT or MRI of the patient with fiducial markers.

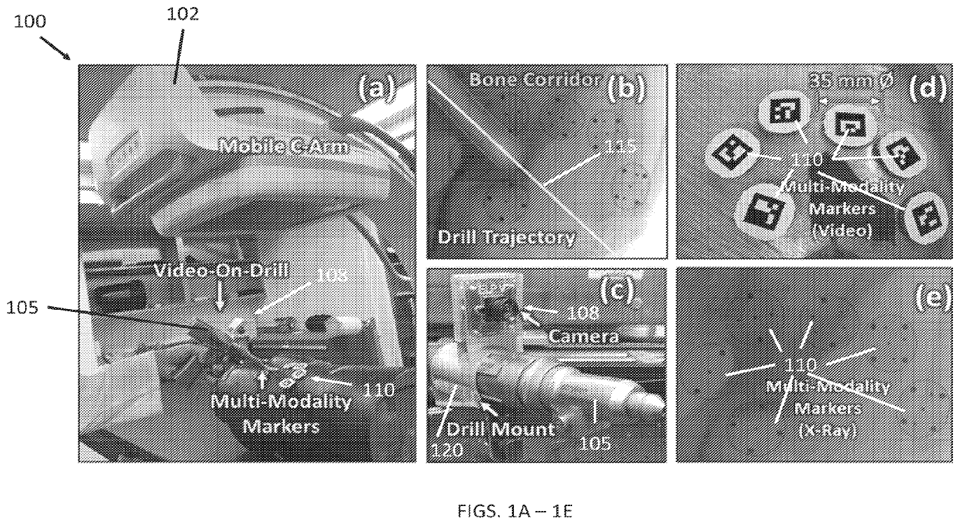

Some embodiments use a miniature optical camera rigidly mounted on the instrument (e.g., surgical drill, biopsy needle, etc.) and multimodality (e.g., optical and radio-opaque) fiducial markers to provide real-time visualization of the drill trajectory in fluoroscopy and/or CT images, as discussed below with reference to FIGS. 1A-1E. In some embodiments, a mobile C-arm is used as the x-ray imaging system, and in other embodiments other x-ray imaging systems (e.g. robotic C-arm, O-arm, etc.) may also be envisioned. Similarly, while some embodiments describe automatic registration to a preoperative CT volume, the disclosed methods are also applicable to other embodiments using alternative forms of preoperative imaging, such as magnetic resonance imaging (MRI) and cone-beam computed tomography (CBCT).

FIGS. 1A-1E illustrate an example embodiment of a Video-on-drill system 100, with a mobile X-ray C-arm 102, drill 105, video camera 108 on the drill, and fiducial markers 110. FIG. 1A shows an illustration of the basic concept, including freehand positioning of the drill 105, placement of markers 110 about the surgical field, and co-registration of the markers 110. FIG. 1B shows the visualization and guidance via 3D-2D registration of the drill axis trajectory 115 in fluoroscopy. FIG. 1C shows a 3D-printed drill mount 120 to rigidly hold the video camera 108. FIG. 1D shows a video scene of the markers 110 placed about the drill entry point. FIG. 1E shows the same markers 110 as seen in C-arm 102 x-ray fluoroscopy.

Some embodiments of the invention are operated in a “Video-Fluoro” mode of visualization and guidance. In this mode (referred to as “Video-Fluoro”), fluoroscopic views are overlaid with the registered drill axis trajectory in real-time according to the current pose of the drill. This mode of navigation allows the surgeon to adjust freehand drill trajectory in real-time to align with the planned trajectory, with the background anatomical scene providing important visual context. Visualization in multiple augmented fluoroscopic views gives reliable 3D reckoning of the scene (normally done mentally, requiring years of training in understanding the complex morphology of the pelvis in projection views). This mode of operation is compatible for patients with and without diagnostic 3D imaging.

Some embodiments of the invention are operated in a “Video-CT” mode of visualization and guidance. This mode of operation (referred to as “Video-CT”) requires a preoperative image and provides 3D navigation analogous to common surgical tracking systems. The CT image (and fluoroscopic views) is overlaid with the drill axis trajectory (rendered in real-time according to the pose of the drill) along with preoperative planning information (e.g. acceptance corridors) to aid the surgeon in determining whether the given trajectory conforms to the bone corridor.

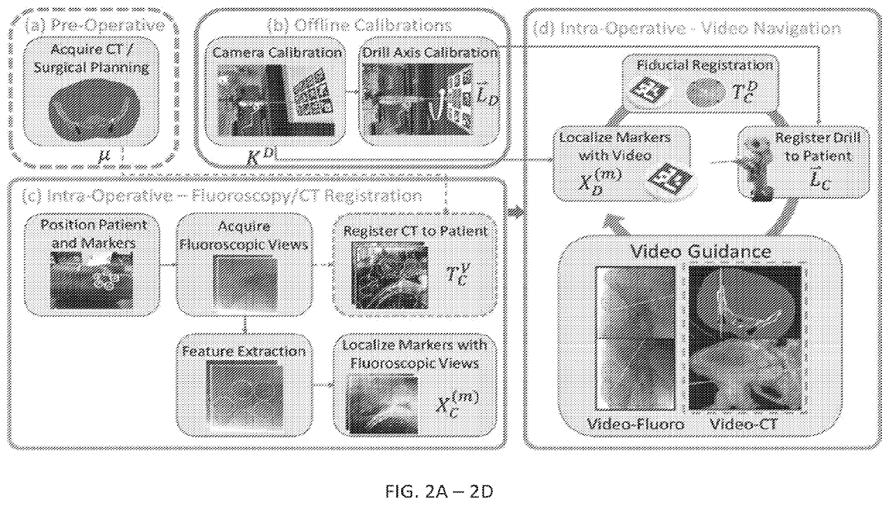

Workflow. The workflow of some embodiments of a drill guidance system is now described with reference to FIGS. 2A-2D. If a preoperative CT of the patient is available (FIG. 2A), then the process enables “Video-Fluoro” and/or “Video-CT” navigation (FIG. 2D). If pre-operative CT is not available, then the system provides Video-Fluoro augmentation only. The workflow includes offline (e.g., pre-operative) and intraoperative steps as follows.

The offline workflow includes two main steps in some embodiments. The first is calibration of the drill-mounted camera (FIG. 2B), which is a one-time, offline process to correct camera-lens distortion. The second is a drill axis calibration which computes the rigid geometric transformation between the camera and the surgical drill axis. This is a relatively quick step that could be performed offline, or alternatively, in the operating room (OR) at the beginning of the case, or intraoperatively if the camera mount is swapped between drills.

The intraoperative workflow also includes two main steps in some embodiments. The first (FIG. 2C) is to place multimodal markers about the surgical field such that at least 3 are in the fluoroscopy field of view (FOV). Note that the markers need not be rigidly affixed with respect to each other or the patient, since the registration is updated with each fluoroscopic view. The drill itself does not need to be visible in the fluoroscopic view; rather, the drill-mounted camera need only have clear view of the markers as present in the fluoroscopic image.

The second step (FIG. 2D) involves continuous, real-time registration of the drill trajectory with respect to the patient (i.e., to fluoroscopy and/or CT) via the video scene. Note that the trajectory overlay shows the drill axis (not the K-wire tip), thereby serving as a guide for the challenging initial step of finding an acceptable entry point and trajectory orientation.

FIGS. 2A-2D are a flowchart illustrating the workflow and algorithms of some embodiments for surgical guidance with a drill-mounted video camera. If available, preoperative CT (FIG. 2A) is registered to the patient (dashed lines) via 3D-2D registration to intraoperative fluoroscopy, allowing “Video-CT” guidance. FIG. 2B shows offline calibration of the camera and drill axis. FIGS. 2C and 2D show intraoperative registration: FIG. 2C shows 3D-2D registration to localize marker poses in C-arm fluoroscopy and register preoperative CT to the patient; and FIG. 2D shows a continuous, real-time loop that registers the drill axis to fluoroscopy and/or CT via the video scene.

Some embodiments employ an algorithm for displaying a drill trajectory in imaging coordinates. For example, the registration of a surgical drill axis, a video camera, an x-ray imaging system, and any available preoperative images is computed in some embodiments as follows. A video camera mounted onto a surgical drill is calibrated with respect to the drill axis. Multimodal markers identifiable in both video and x-ray images are placed about the surgical field and co-registered by feature correspondences. If available, a preoperative CT can also be co-registered by 3D-2D image registration. Real-time guidance is achieved by overlay of the drill axis on fluoroscopy and/or CT images.

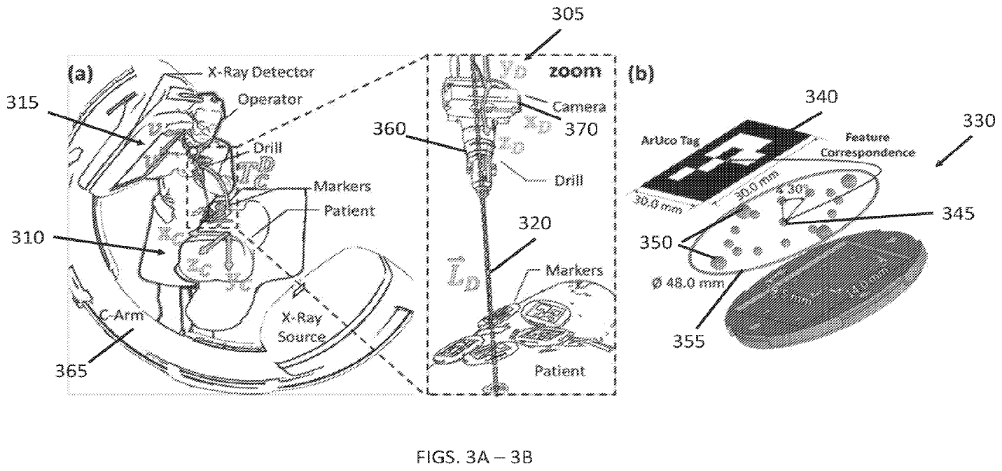

FIG. 3A further illustrates the coordinate systems and transforms, including: the drill camera 3D coordinate frame (denoted D, X_{D}, Y_{D}, Z_{D}) 305, the C-arm 3D coordinate frame 310 (denoted C, X_{C}, Y_{C}, Z_{C}), and the preoperative CT 3D coordinate frame (denoted v, u) 315. The zoomed-in view shows the drill camera (and frame D) in relation to the drill axis ( [symbol] _{D}) 320.

FIG. 3B illustrates a schematic of multimodal marker 330 features for some embodiments visible in both video (e.g., the center of the ArUco tag 340) and fluoroscopy (a central BB 345). Additional BBs 350 and wires 355 encode individual markers and provide a basis for 3D-2D registration between the marker design (κ) and fluoroscopy.

FIGS. 3A and 3B illustrate coordinate frames and multimodal marker design. In FIG. 3A, coordinate frames for the video-guided drill setup, including the drill frame 305 (D) for the drill camera 360 and the C-arm coordinate frame 310 (C) for the C-arm 365. The zoomed-in view shows the drill camera 370 (and frame D 305) in relation to the drill axis (\vec{L}_{D}) 320. FIG. 3B shows a schematic of multimodal marker 330 features visible in both video (center of the ArUco tag 340) and fluoroscopy (central BB 345). Additional BBs 350 and wires 355 encode individual markers 330 and provide a basis for 3D-2D registration between the marker design (κ) and fluoroscopy.

Table 1 summarizes notation for relevant coordinate frames, transforms, and variables for video-based surgical drill guidance and notation, with 3D vectors denoted in uppercase and 2D vectors denoted in lowercase. The Coordinate frames are: D (for drill camera), C (for the C-arm), and V (for preoperative CT volume). Multimodal markers (m=1, . . . , M) are co-registered between the video frame, one or more fluoroscopic images at view angles (θ), and the CT volume, via 3D-2D registration.

TABLE 1
System Parameters

| | |
|---|---|
| θ | Projection view angle for a C-arm fluoroscopy frame |
| m | Index for a particular multimodal marker (m: 1 . . . M) |
| κ | 3D mesh representation of radio-opaque marker features |
| μ | Preoperative CT volume |

Drill Camera Coordinate Frame (D)

| | |
|---|---|
| K^{D} | Drill camera calibration matrix |
| X_{D}^{(m)} | 3D feature point for marker m in drill camera 3D |
| | coordinate frame |
| x_{D}^{(m)} | 2D feature point for marker m in drill camera 2D image |
| | plane |
| P_{D}^{(m)} | Pose of marker m in the drill camera 3D coordinate frame |
| [symbol] _{D} | Drill axis in drill camera 3D coordinate frame |

C-arm Coordinate Frame (C)

| | |
|---|---|
| [symbol] ^{C}(θ) | C-arm projection matrix for fluoroscopy frame at view |
| | angle θ |
| X_{C}^{(m)} | 3D feature point for marker m in the C-arm 3D |
| | coordinate frame |
| x_{C}^{(m,θ)} | 2D feature point for marker m in the C-arm 2D image |
| | plane for a fluoroscopy frame at view angle θ |
| p_{C}^{(m)} | Pose of marker m in the C-arm 3D coordinate frame |
| T_{C}^{D} | Transformation from the drill camera to C-arm 3D |
| | coordinate frames |
| [symbol] _{C} | Drill axis in the C-arm 3D coordinate frame |
| [symbol] | Drill axis projected to the C-arm 2D image plane |
| | (i.e., 2D fluoroscopy frame) |

Preoperative CT Coordinate Frame (V)

| | |
|---|---|
| T_{C}^{V} | Transformation from the preoperative CT to C-arm 3D |
| | coordinate frames |
| [symbol] _{V} | Drill axis in the preoperative CT coordinate frame |

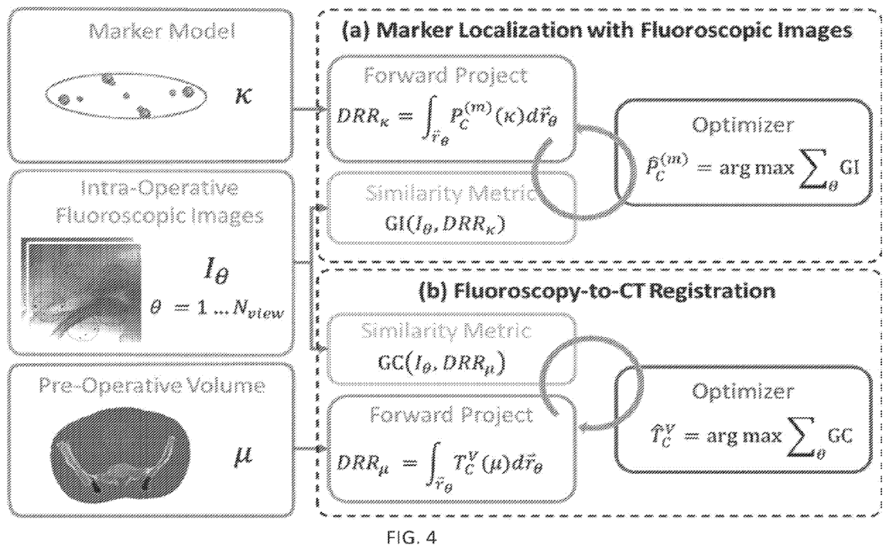

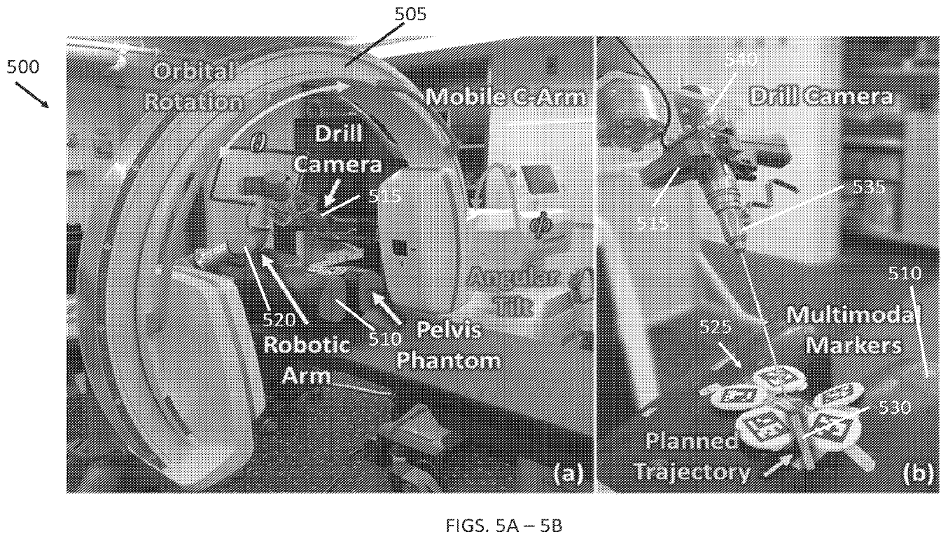

FIG. 4 illustrates a flowchart of some embodiments for the 3D-2D registration pipeline of intraoperative fluoroscopy to (a) the markers and (b) the preoperative CT. The equations within the figure describe an example embodiment of each block of the algorithm.

### Instrument Axis Calibration

In some embodiments, camera calibration is performed to estimate the intrinsic parameters of the camera (pinhole model) and distortion coefficients of the lens. The calibration is performed in some embodiments using well-known resectioning techniques such as Zhang's method³⁰ or Tsai's method³¹. The instrument (e.g., drill) axis is then calibrated to the drill camera coordinate frame ( [symbol] _{D}). The examples below describe embodiments for a surgical drill, though other embodiments with other types of instruments are also envisioned.

A description of one embodiment for camera calibration to a drill is here described. The intrinsic parameters are represented by the camera calibration matrix (K^{D}) consisting of principal points (a₀, b₀) and focal lengths (fₐ, f_{b}):

$${K}^{D} = [ \begin{matrix}{f}_{a} & 0 & {a}_{0} \\ 0 & {f}_{b} & {b}_{0} \\ 0 & 0 & 1\end{matrix} ] \qquad ( 1 a )$$

Calibration is performed in some embodiments using the resectioning method of Zhang et al.³⁰ using multiple images of a planar checkboard to obtain a closed form solution for the intrinsic parameters of the camera. The resulting parameters are then jointly refined along with the distortion coefficients by least—squares minimization of the reprojection error across all image points. Lens distortion is modeled in some embodiments using the Brown-Conrady even-order polynomial model³⁰ to remap image points (x̆_{D}) onto distortion-corrected image points (x_{D}) with a model describing both radial (k₁, k₂) and tangential distortion (p₁, p₂) effects:

a=ă+(ă−a₀)(k₁r²+k₂r⁴)+2p₁(ă−a₀)(b̆−b₀)+p₂[r+2(ă−a₀)²]

b=b̆+(b̆−b₀)(k₁r²+k₂r⁴)+2p₁(ă−a₀)(b̆−b₀)+p₁[r+2(b̆−b₀)²]

r=\sqrt{(ă−a₀)²+(b̆−b₀)²} (1b)

where (a, b) are components of the distortion-corrected image point x_{D}, (ă, b̆) are components of the uncorrected image point [symbol] , and (a₀, b₀) are the principal points shown in Eq. (1a) approximating the center of distortion.

The orientation of the drill-axis in the drill camera coordinate frame (\vec{L}_{D}) is solved in some embodiments using a calibration jig constructed to freely spin about the drill axis. In some embodiments, the jig includes a drill sleeve centered on an ArUco board³⁴ containing a square 3×3 grid of ArUco tags (with inner marker tag removed). The calibration jig is attached to the instrument (e.g. K-wire, drill bit, screw, etc.) and rotated along the axis of the instrument. As the jig (drill sleeve) rotates about the drill axis, the pose of the ArUco board. in the 3D frame of the camera is estimated in multiple images using the Perspective-N-Points (PNP) algorithm. For circular motion of the jig, pose estimation from multiple camera images yields a 3D cylindrical point cloud, and a generalized RANSAC-based cylindrical fit (MLESAC⁴²) is computed to obtain the central axis of the point cloud—i.e., the drill (or other instrument) axis (\vec{L}_{D}). Due to constraints on motion of the calibration jig, the cylindrical axis represents the surgical drill axis in the drill camera coordinate frame.

### Video-to-Fluoroscopy Registration

Video Features. In some embodiments, video images are registered to the fluoroscopic scene through localization and registration of point-based feature correspondences within the multimodal markers. Video features are realized through a variety of vision-based fiducial systems reported in the prior art (e.g. ArUco, AprilTag, ARToolKit). In some embodiments, for example, ArUco tags are used.

Image-based 3D-2D registration methods are used in some embodiments to estimate the. pose of the markers from fluoroscopic images in the C-arm coordinate frame (C) through image similarity metrics. In some embodiments, a calibrated C-arm is used with projective relationship [symbol] ^{C} relating 3D points in the C-arm frame (X_{C}) to 2D fluoroscopic image points on the C-arm detector plane (x_{C}). To extract marker pose from fluoroscopic images, some embodiments perform 3D-2D “known-component” registration (KC-Reg)³³. The general framework for KC-Reg is as shown in panel (a) of FIG. 4. A generic marker model (κ) representing the radio-opaque features of the marker is iteratively transformed in some embodiments to optimize the image similarity between digitally reconstructed radiographs (DRR_{κ}) and one or more intraoperative fluoroscopic images (I_{θ}). Fluoroscopic views were selected such that markers were not overlapping in projection data.

A description of an example embodiment for video-to-fluoroscopy registration here follows.

Video images were registered to the fluoroscopic scene through localization and registration of the point-based feature correspondences of the multimodality markers (discussed in more detail below). For the ArUco tags, the 3D pose of each marker in the drill camera coordinate frame (P_{D}^{(m)}) is estimated via well-known camera pose estimation techniques (for example, the PNP algorithm) for m=1, . . . , M markers. The translational component of the resulting pose estimate is extracted to represent the central marker feature point in the drill camera coordinate frame [X_{D}^{(m)}].

Similar to PNP, 3D-2D registration methods use fluoroscopic images to estimate the pose of the markers in the C-arm coordinate frame (C) through image similarity metrics rather than explicit point-to-point correspondences. In this work, a calibrated C-arm with projective relationship [symbol] ^{C} related 3D points in the C-arm frame (X_{C}) to 2D fluoroscopic image points on the C-arm detector plane (x_{C}) (in homogenous coordinates) by:

$$( \begin{matrix}{x}_{C} \\ 1\end{matrix} ) = {𝒫}^{C} ( \begin{matrix}{X}_{C} \\ 1\end{matrix} ) \qquad ( 2 a )$$

The projection matrix was obtained by standard C-arm geometric calibration methods⁴³ and is defined by:

[symbol]

^{C}=K^{C}[ [symbol] _{Θ} [symbol] _{]}

$${K}^{D} = [ \begin{matrix}SDD & 0 & {u}_{0} \\ 0 & SDD & {v}_{0} \\ 0 & 0 & 1\end{matrix} ] \qquad ( 2 b )$$

The intrinsic matrix K^{C} describes the geometric relationship between the C-arm source and detector as described by the source-to-detector distance (SDD) and the piercing point (u₀, v₀) representing the position of the orthogonal ray between the source and detector plane. The extrinsic parameters ( [symbol] _{Θ} [symbol] ]) describe the pose of the C-arm source-detector assembly for a fluoroscopy frame at view angle θ in a common coordinate frame (referred to as the C-arm coordinate frame C).

Optional Pre-Processing, Video and/or fluoroscopic views are pre-processed. in some embodiments for better performance and to improve robustness of the registration (e.g., noise reduction, edge enhancement, etc.). For best performance, fluoroscopic views are selected in some embodiments such that markers are not overlapping in projection data. To improve robustness of the registration, fluoroscopic images are pre-processed in some embodiments to isolate the marker features from background anatomy (e.g., with morphological top-hat filtering and gamma expansion) and masked to mitigate interference from neighboring marker features.

Similarity Metric. The similarity between a fixed fluoroscopic image (I_{fixed}) and its corresponding moving DRR (I_{moving}) is computed in some embodiments through a variety of metrics (e.g. cross-correlation, gradient-based similarity). In some embodiments, the gradient information (GI) similarity metric is used as it has been shown to be robust in the presence of strong gradient magnitudes not present in both images.^{34,35} To solve for the pose of marker m, an objective function that maximizes the cumulative GI across θ=1, . . . , N_{view} fluoroscopic views was defined as:

$${\hat{P}}_{C}^{( m )} = arg {max}_{{P}_{C}^{( m )}} {\sum }_{θ} GI ( {I}_{θ} , {\int }_{{\vec{r}}_{θ}} {P}_{C}^{( m )} ( κ ) d {\vec{r}}_{θ} ) \qquad ( 3 )$$

where the moving DRR is computed by integrating rays ( [symbol] ) along the transformed mesh volume P_{C}^{(m)}(κ) according to the C-arm gantry pose at view θ.

Optimization Method. In some embodiments, optimization of the pose estimate \hat{P}_{C}^{(m)} is performed with any general optimizer. For example, the pose estimate \hat{P}_{C}^{(m)} was optimized using the covariance matrix adaptive evolution strategy (CMA-ES) algorithm³⁶ from which the translational component was extracted to represent the central marker feature point in the C-arm coordinate frame [\hat{X}_{C}^{(m)}]. Prior to optimization, the pose was initialized using features extracted during marker detection. For each multimodal marker m, an initial estimate of the central 3D feature point in the C-arm frame [\hat{X}_{C}^{(m)}] was obtained by first backprojecting a ray from the corresponding 2D image feature point in homogenous coordinates (referred to in bold type as x_{C}^{(m,θ)}) toward the x-ray source. The backprojected ray was estimated for each fluoroscopic view θ, from which an initial estimate of the 3D feature point could be reconstructed by:

$${\hat{X}}_{C}^{( m )} = \frac{1}{{N}_{view}} {\sum }_{θ = 1}^{{N}_{view}} \frac{{{λ}^{( m , θ )} ( {K}^{C} {ℛ}_{θ} )}^{- 1} {x}_{C}^{( m , θ )}}{‖ {( {K}^{C} {ℛ}_{θ} )}^{- 1} {x}_{C}^{( m , θ )} ‖} + ( - {ℛ}_{θ}^{- 1} {𝒯}_{θ} ) \qquad ( 4 a )$$

$$where$$

$${λ}^{( m , θ )} = \frac{SDD}{{ℳ}^{( m , θ )}} \qquad ( 4 b )$$

describes the estimated distance along the backprojected source-detector ray. The magnification [symbol] was estimated for each marker m in each fluoroscopic view using the perspective relationship between the diameter of a circle and the major axis length of its elliptical projection. Once the 3D position of each marker was initialized, a rotational initialization was performed with a planar fit of the global marker arrangement. The computed plane normal was used as an initial estimate of the out-of-plane axis for each marker.

Once initialized, the CMA-ES optimization for each marker was performed following a coarse-to- fine multiresolution strategy. First, a coarse multi-start was performed in which KC-Reg was reinitialized 7 times with 45° rotational offsets to initialize the rotational pose of the marker. A refinement was then computed at fine resolution to obtain the final marker pose estimate (\hat{P}_{C}^{(m)}). The coarse stage was carried out at 1×1 mm² pixel size, with a total population size of 350 and initial standard deviation of σ=10 mm (and 10°). The refinement was performed at 0.5×0.5 mm² pixel size with a population size of 50 and initial standard deviation of σ=5 mm (and 5°).

With corresponding point estimates obtained in both the drill camera (D) and C-arm (C) coordinate frames, a transformation between the two was estimated using point-based registration described by Horn et al.³⁷. The resulting video-to-fluoroscopy transform (T_{C}^{D}) was used to represent the surgical drill axis in the C-arm coordinate frame:

[symbol]

_{C}=T_{C}^{D} [symbol] _{D} (5a)

and its projection on the C-arm detector plane:

[symbol]

_{C}= [symbol] ^{C} [symbol] (5b)

Visualization/Display. Augmentation of fluoroscopic views with [symbol] _{C} realizes the “Video-Fluoro” navigation mode of some embodiments. During instrument guidance, only the localization in the camera images is continuously updated, not fluoroscopy frames, so long as the markers are not perturbed relative to anatomy. If perturbed, the video-to-fluoroscopy registration is updated in some embodiments by acquisition of one or more fluoro shots.

### Fluoroscopy-to-CT Registration

Intraoperative fluoroscopy is used in some embodiments to register the patient to a preoperative CT (or intraoperative CBCT) volume using 3D-2D registration³⁴⁻³⁸. The workflow for patient registration in some embodiments is illustrated in panel (b) of FIG. 4. The CT volume (μ) is iteratively transformed to optimize similarity between fluoroscopic images (I_{θ}) and forward projections of the rigidly transformed CT (DRR_{μ}) using an objective function similar to Eq. 3.

In some embodiments, the gradient correlation (GC) metric is used for CT registration because it is independent of absolute gradient magnitudes (unlike GI) making it more robust for registration between images in which corresponding anatomical gradients may differ due to differences in imaging techniques or mismatch in image content (e.g., tools in the fluoroscopic scene that are not in the CT).³⁸ For marker localization, GI is observed to perform well due to its relative insensitivity to gradients from surrounding anatomy. In some embodiments, a coarse-to-fine multiresolution strategy is employed, with a coarse multi-start consisting of 7 reinitializations (each with a 5° rotational offset) at a resolution of 2×2 mm² pixel size, a total population size of 700, and initial standard deviation of σ=10 mm (and 10°). A refinement is subsequently computed at higher image resolution (0.5×0.5 mm² pixel size) with a population size of 50 and initial standard deviation of σ=5 mm (and 5°).

The resulting CT-to-fluoroscopy transform (T_{C}^{V}) in some embodiments is used to transform the surgical drill axis into the preoperative CT coordinate frame as:

[symbol]

ᵥ=(T_{C}^{V})⁻¹T_{C}^{D} [symbol] _{D} (6)

Augmentation of the CT image (e.g., orthogonal slices, MIPs, or volume renderings) realizes the “Video-CT” navigation mode of some embodiments in which the instrument trajectory is visualized in the 3D CT image.

In some embodiments, fluoroscopy-to-CT registration operates under the assumption of rigidity, which is appropriate in the context of simple fractures. The drill-mounted video concept is integrated in some embodiments with recent 3D-2D registration methods that address challenges of joint dislocation⁴⁰ and multi-body comminuted, displaced. fractures.^{44,45} Such integration extends the potential applicability across a filler range of pelvic trauma surgery.

### Experimental Evaluation: Phantom Study Example

FIGS. 5A and 5B illustrate an example embodiment of an experimental setup for evaluating the performance of the video-guided drill system in terms of the accuracy of drill axis registration and guidance along K-wire trajectories common in pelvic trauma surgery. Other embodiments are applicable to a broader range of image-guided procedures (e.g., hip, knee, and wrist surgery) that conventionally use fluoroscopy to guide instrument placement.

FIG. 5A illustrates the video-based drill guidance imaging setup 500, showing the C-arm 505, pelvis phantom 510, and video-drill 515 held by a robotic positioner 520. FIG. 5B shows a detail view of the drill camera 515 and the markers 525. The markers 525 are placed on the pelvis phantom 510 about the entry for the anterior inferior iliac spine (AIIS) to posterior superior iliac spine (PSIS) trajectory 530.

An anthropomorphic pelvis phantom 510 composed of a natural adult skeleton in tissue-equivalent plastic (Rando®, The Phantom Lab, Greenwich NY) was used. A UR3e robotic arm 520 (Universal Robotics, Odense, Denmark) was used as a drill holder throughout the experiments, but a robot is not required in other embodiments. FIG. 5B shows an embodiment for the drill guidance system consisting of a Stryker System 6 (Kalamazoo, MI) surgical drill 535 with a pin collet for 3 mm diameter K-wires and a Logitech C900 USB camera 515. The camera 515 was rigidly attached to the drill body using a custom 3D-printed chassis 540 that positioned the camera 515 with clear view of the drill axis and surgical scene. Because all experiments were performed in an operating room (OR) laboratory (i.e., decommissioned clinical OR), lighting conditions approximated realistic OR lighting in terms of ambience with or without an overhead OR light (not shown in FIG. 5).

The drill camera 515 was aligned with respect to the anterior inferior iliac spine (AIIS) to posterior superior iliac spine (PSIS) trajectory in the left hip. Five markers 525 were placed about the planned entry site. The robotic arm 520 was used to position the drill camera 515 at a distance of ˜20 cm from the surface of the phantom 510 to emulate realistic surgical drill positioning. Nine camera poses were sampled about the initial planned trajectory 530 to measure the sensitivity of fiducial registration error to various perspectives.

A mobile isocentric C-arm 505 (Cios Spin, Siemens Healthineers, Forcheim, Germany) was used to acquire fluoroscopic images (for 3D-2D registration) and CBCT (for truth definition). CBCT images were acquired with 400 projections over a 195° semi-circular orbit and reconstructed on a 0.512×0.512×0.512 mm³ voxel grid with a standard bone kernel. An initial CBCT scan (110 kV, 350 mAs) was acquired—with only the pelvis phantom 510 and marker 525 arrangement present in the field of view (FOV). The projections from the scan where markers 525 were not overlapping (an orbital arc of θ=−20° to 70°) were selected as candidate views for solving 3D-2D marker localization. The resulting CBCT reconstruction was used to segment the central BB position for each marker 525 as truth definition. Single fluoroscopic views (100 kV, ˜0.9 mAs) were collected at common clinical orientations for augmentation in “Video-Fluoro” mode, including: AP view (θ=0°, ϕ=0°), lateral view (θ=90°, ϕ=0°), inlet view (θ=0°, ϕ=−25°), and outlet view (θ=0°, ϕ=30°). The drill camera 515 was then positioned with a 3 mm K-wire extending from the drill tip to the surface of the pelvis phantom 510, and video images of the arrangement of markers 525 were collected. A final CBCT scan (110 kV, 380 mAs) was acquired with the drill camera 515 in the FOV for ground truth definition of the K-wire drill axis.

For evaluation of the “Video-CT” navigation mode, a preoperative CT volume (0.82×0.82×0.5 mm³ voxel grid) of the pelvis phantom 510 was acquired (SOMATOM Definition, Siemens, Erlangen Germany). K-wire trajectories were automatically planned in the preoperative CT using the atlas-based planning method in Goerres et al.³⁹ and Han et al.⁴⁰ Acceptance volumes interior to bone corridors were created for common K-wire trajectories, including: AIIS-to-PSIS, superior ramus (SR), and iliac crest (IC) to posterior column (PC). Acceptance volumes were used to visualize and evaluate drill axis conformance within pelvic bone corridors.

Table 2 provides a summary of figures of merit for assessing the accuracy of individual registration methods and end-to-end system performance. The performance of video-to-fluoroscopy registration was quantified in terms of errors related to 3D marker localization in the drill camera (D) and C-arm (C) coordinate frames.

TABLE 2
Video-to-Fluoroscopy Registration

| | |
|---|---|
| FRE^{(m)} | Norm of error in 3D localization of marker m from |
| | video images |
| Δ_{D}^{(m)} | In-plane and out-of-plane translational error components |
| | in 3D localization of marker m from video images |
| δ_{C}^{(m)} | Norm of error in 3D localization of marker m from |
| | fluoroscopic images |
| Δ_{C}^{(m)} | In-plane and out-of-plane translational error components |
| | in 3D localization of marker m from fluoroscopic images |

Fluoroscopy-to-CT Registration

| | |
|---|---|
| Γ_{V} | Fluoroscopy-to-CT registration difference transform |
| Γ_{V}^{Δ} | In-plane and out-of-plane translational error components |
| | in fluoroscopy-to-CT registration |
| Γ_{V}^{Φ} | In-plane and out-of-plane rotational error components in |
| | fluoroscopy-to-CT registration |
| δ_{V} | Norm of translational error in fluoroscopy-to-CT |
| | registration |

Drill Axis Registration

| | |
|---|---|
| TRE_{X} | Norm of translational error between predicted and |
| | reference drill axes |
| TRE_{φ} | Angular skew between predicted and reference drill axes |

The positional estimate for each marker in the camera frame [\hat{X}_{D}^{(m)}] was evaluated with respect to the truth definition (points defined in CBCT) and quantified in terms of fiducial registration error (FRE):

FRE=||\hat{T}_{C}^{D}\hat{X}_{D}^{(m)-}X_{C}^{(m,true)}|| (7a)

where X_{C}^{(m,true)} represents the true location of marker m in the C-arm frame and \hat{T}_{C}^{D} is an estimate of the true camera-to-C-arm transform derived from point-based registration with all markers. The term \hat{T}_{C}^{D}\hat{X}_{D}^{(m)} therefore is the estimated location of marker m in the C-arm frame. Fiducial errors were further decomposed into in-plane and depth errors with respect to the drill camera coordinate frame (Δ_{D}) as:

Δ_{D}= [symbol] _{D}^{C}(\hat{T}_{C}^{D}\hat{X}_{D}^{(m)}−X_{C}^{(m,true)}) (7b)

Estimation of [symbol] _{D}^{C} (the rotation matrix from the C-arm to the drill camera coordinate frame) was performed independently of the fiducials by solving the rotation between the calibrated drill axis ( [symbol] _{D}) and the truth definition for the drill axis segmented from CBCT (referred to as the reference drill axis, [symbol] _{C}^{(true)}).

Marker localization errors in the C-arm frame (C) were estimated with reference to the ground truth marker locations (X_{C}^{(m,true)}). The 3D-2D registration of each marker 525 was solved using N_{view}=1−3 fluoroscopic views, selected from the subset of candidate projections mentioned earlier. For N_{view}>1, selected views were chosen such that they spanned (in total) a 30° arc with equiangular spacing. Accuracy was assessed in terms of the norm of the translational error (δ_{C}):

δ_{D}^{(m)}=||\hat{X}_{C}^{(m)}−X_{C}^{(m,true)}|| (7c)

For registrations based on a single fluoroscopic view (N_{view}=1), translational errors were further examined with respect to in-plane (parallel to the detector plane) and out-of-plane (depth) components (referred to as Δ_{C}).

Fluoroscopy-to-CT registration was evaluated over the same set of fluoroscopic views used during marker localization for N_{view}=1 and 2 (over a 30< arc). To evaluate accuracy, truth was defined from a large number (N_{view}=15) of fluoroscopic views to solve the true 3D-2D patient registration (referred to as [symbol] ^{V}), selecting distinct views for registration and truth definition to mitigate bias. Performance was calculated in terms of the difference transform (Γ_{V}) between the registration result and the truth definition by:

Γ_{V}=\hat{T}_{C}^{V}( [symbol] _{C}^{V})⁻¹ (8)

The difference transform was further decomposed into translational (Γ_{V}^{Δ}) and rotational error components (Γ_{V}^{φ}), from which the norm of the translational error (δ_{V}) was also computed.

End-to-end system performance (registration accuracy) was evaluated by comparison of the predicted drill axis ( [symbol] _{C}) with the reference drill axis isolated from CBCT ( [symbol] _{C}^{(true)}). To correct for possible mismatch between the reference trajectory and the automatically planned trajectory, a transformation was applied to both the predicted and reference drill axes. This transformation places the drill 535 within the context of the bone corridor, as would be done in clinical use, while preserving the relative errors between the predicted and reference trajectories. The predicted and reference drill axes were separated into translational (τ_{C}) and rotational (ρ_{C}) components from which errors in positional and angular measurements could be computed, respectively, as:

$${TRE}_{x} = ‖ {\hat{τ}}_{C} - {τ}_{C}^{( true )} ‖ \qquad ( 9 a )$$

$${TRE}_{φ} = {cos}^{- 1} \frac{{\hat{ρ}}_{C} \cdot {ρ}_{C}^{( true )}}{‖ {\hat{ρ}}_{C} \cdot {ρ}_{C}^{( true )} ‖} \qquad ( 9 b )$$

where the hat operator denotes components of the predicted drill axis. Here, the rotational components (ρ_{C}) are given by a vector describing the direction of the drill axis, and the translational components (τ_{C}) describe a point along the drill axis. Since the drill was aligned with respect to a planned bone corridor (AIIS-to-PSIS), the translational component was set to correspond to the bone corridor entry point. The entry point was computed as the intersection of the predicted and reference drill axes with the surface of the planned acceptance volume. All registration errors reported were computed in the CT coordinate frame (V). The reference trajectory in the CT coordinate frame was estimated using the fluoroscopy-to-CT truth definition

( [symbol] _{V}^{(true)}=( [symbol] _{C}^{V})⁻¹ [symbol] _{C}^{(true)}).

To evaluate end-to-end system performance in a broader context, the conformance of drill axis trajectories was evaluated relative to multiple pelvic bone corridors. For each bone corridor, a mesh of the planned acceptance volume was created to represent the cortical bone surface. The acceptability of a resulting trajectory was measured by first computing the entry and exit point at which the given trajectory intersects the cortical bone surface. The resulting path from entry to exit point was equidistantly sampled along the trajectory, and the distance from each sample to the nearest cortical bone surface point was calculated. Measurements less than zero (or less than the radius of the K-wire) suggest a breach of the bone cortex, An ensemble of drill axis trajectories was simulated as a dispersion of trajectories about the reference trajectory according to the median positional and angular TRE (Eq. 9). In addition to the AIIS-to-PSIS trajectory, the conformance was analyzed relative to the SR and IC-PC bone corridors.

### Accuracy of Video-to-Fluoroscopy Registration

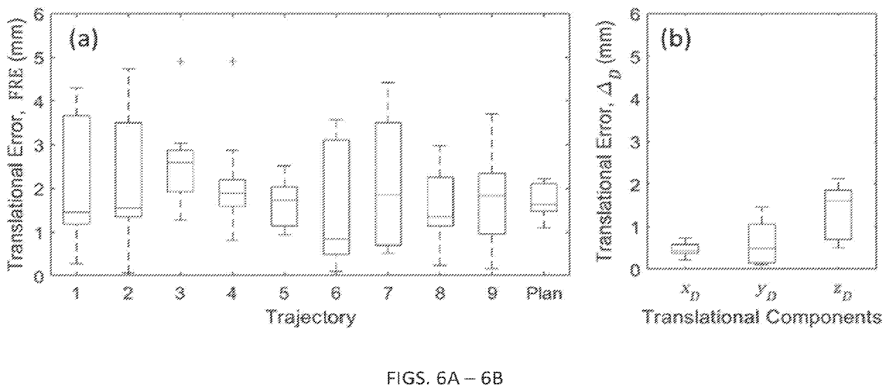

FIGS. 6A and 6B show registration error for marker localization by the camera. FIG. 6A shows camera-to-C-arm fiducial registration errors (FRE) across nine trajectories sampled freehand about the AIIS-PSIS trajectory plan. Trajectories were selected by radially sampling poses about the center of the marker arrangement. Also shown is the FRE for the actual planned trajectory [denoted in (a) as Plan]. FIG. 6B shows, for the planned trajectory, how errors were further decomposed into in-plane (xD, yD) and out-of-plane (zD) camera localization errors (ΔD) in the drill camera coordinate system.

FIG. 6A summarizes the accuracy of 3D marker localization from video images-FRE as in Eq. (7a) pooled over all (M=5) markers. Measurements are shown for the planned ASIS-PSIS trajectory as well as nine camera poses sampled about the planned trajectory to examine how localization errors in the video scene translate to errors in fiducial registration. Within a given camera pose, the registration performance exhibited considerable variation between markers (IQR˜3.5 mm) with FRE up to 5 mm in some cases due to poor marker corner detection and pose estimation in images. The median FRE, however, was fairly consistent across all camera poses, with median error of 1.5 mm (IQR=0.42 mm) for the nine trajectories sampled about the plan.

Trajectories were selected by radially sampling poses about the center of the marker arrangement. Also shown is the FRE for the actual planned trajectory [denoted in (a) as Plan].

FIG. 6B illustrates the localization errors with respect to the drill camera coordinate frame—Δ_{D} as in Eq. (7b), decomposed in terms of in-plane and out-of-plane translational components for the planned trajectory. The translational errors (x_{D}, y_{D}) are parallel to the camera image plane, and z_{D} represents out-of-plane errors perpendicular to the camera plane. In-plane translations exhibited median error of 0.46 mm (IQR=0.52 mm), and out-of-plane errors were notably higher, with median error of 1.6 mm (IQR=1.2 mm).

Overall, the results demonstrate consistent FRE across the sampled trajectories, suggesting reasonable robustness in localizing markers from video images from a broad variety of camera poses. Such robustness is important as the surgeon maneuvers the drill about the scene to align with the planned trajectory. The out-of-plane errors (z_{D}) reflect challenges in resolving depth with a monocular camera, addressed in future work incorporating a stereoscopic camera system.

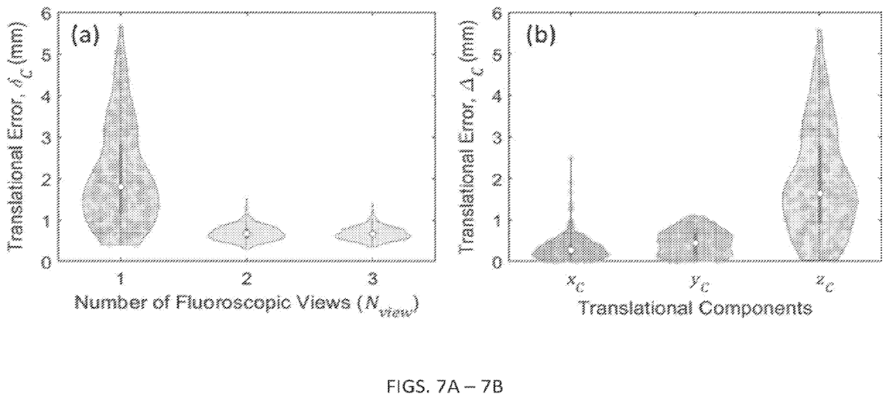

FIGS. 7A and 7B show the errors associated with 3D marker localization from fluoroscopic images, −δ_{C} as in Eq. (7c) pooled over all (M=5) markers. As shown in FIG. 7A, the localization error was notably higher for registrations solved with a single fluoroscopic view, with median error of 1.8 mm (IQR=1.7 mm). Registrations solved with multiple fluoroscopic views improved performance considerably, with N_{view}=2 or 3 (evenly spaced over a 30° arc) giving median error of 0.67 mm (IQR=0.25 mm) or 0.66 mm (IQR=0.20 mm), respectively. The main directional component of such errors is out-of-plane (i.e., along the x-ray source to detector direction), as illustrated in FIG. 7B. Translational errors (Δ_{C}) in (x_{C}, y_{C}) reflect the in-plane component (parallel to the C-arm detector plane), and z_{C} represents out-of-plane errors along the source-detector axis. For the N_{view}=1 case, in-plane errors were ˜0.34 mm (IQR=0.42 mm), compared to ˜1.6 mm (IQR=1.8 mm) out-of-plane, again illustrating the challenge to depth resolution from a single perspective.

FIGS. 7A and 7B show registration error for marker localization with intraoperative fluoroscopy. FIG. 7A shows the norm of translational errors (δc) for registrations solve with N_{view}=1-3. In multi-view scenarios, the selected views were evenly spaced over a 30° arc. FIG. 7B shows component-wise translation errors (Δ_{C}) for single-view (N_{view}=1) marker registration. Errors in x_{C}, y_{C} represent in-plane errors and z_{C} represents errors in out-of-plane depth.

### Accuracy of Fluoroscopy-to-CT Registration

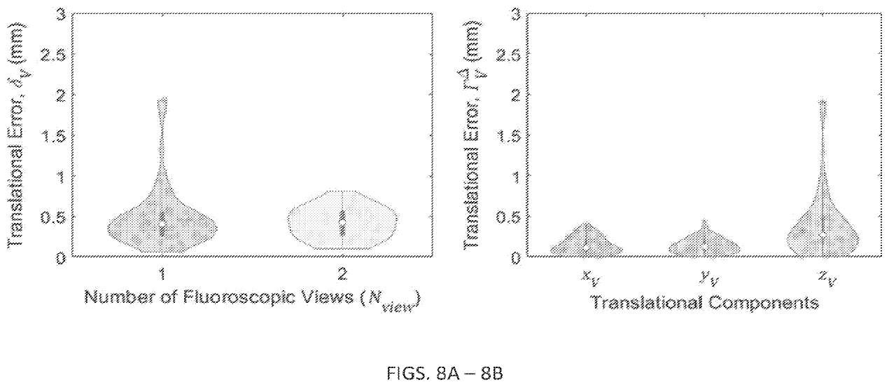

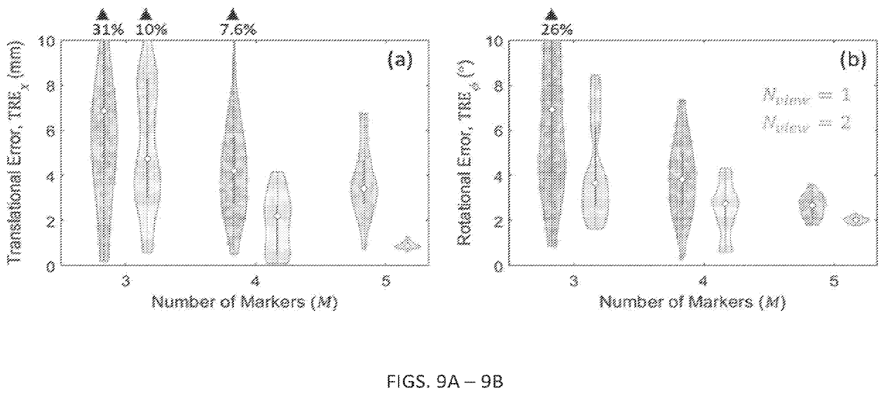

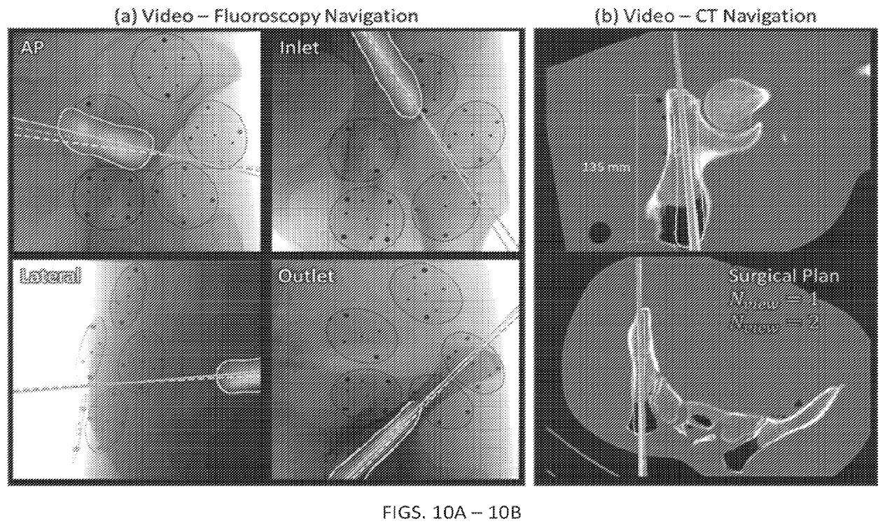

FIGS. 8A and 8B show 3D-2D registration accuracy of preoperative CT to C-arm coordinate frame showing (a) the norm of translational errors (δv) between registrations solved with N_{view}=1 and N_{view}=2 (separated by 30°) and (b) component-wise translation errors (Δ_{V}) for single-view (N_{view}=1) registration. Errors in x_{V} and y_{V} represent in-plane errors, and z_{V} represents errors in out-of-plane depth.

Fluoroscopy-to-CT registration accuracy was computed in terms of the difference in true and estimated 3D transformations—Γ_{V} as in Eq. 8. As shown in FIG. 8A, the translational error norm (δ_{V}) for registrations solved with N_{view}=1 and N_{view}=2 was similar, with median errors of 0.4 mm (0.3 mm IQR) in both cases, noting a few outliers (up to ˜2 mm error) when N_{view}=1. For N_{view}=1, the errors were further separated into translational errors (FIG. 8B) and rotational errors (not shown) relative to the detector plane. Median in-plane translational error (x_{V}, y_{V}) was 0.14 mm (IQR=0.10 mm), and out-of-plane error (z_{V}) was slightly higher [median of 0.39 mm, IQR=0.40 mm) and accounted for the outliers observed in FIG. 8a. In-plane and out-of-plane rotational errors were consistently low (median of 0.07< and 0.03°, respectively).

### Accuracy of Drill Axis Registration.

FIGS. 9A and 9B show performance for end-to-end drill axis localization in the preoperative CT coordinate frame (V). Registration error was assessed as a function of the number of markers in terms of (a) translational error (TREₓ) at the entry point of the planned bone corridor and (b) rotational error (TRE_{φ}) between the predicted and reference drill axes. Registration performance is shown for both N_{view}=1 and 2. The outlier rate (fraction of measurements greater than 10 mm or 10°) is shown above each violin plot.

FIGS. 9A and 9B illustrate positional and rotational TRE [TREₓ and TRE_{φ} in Eq. 9 for the AIIS-to-PSIS trajectory. Registrations were solved using N_{view}=1 or 2 fluoroscopic images within an orbital arc of θ=−20° to 70° with the number of markers, M, varying from 3 to 5 for the arrangement illustrated in FIG. 4. For M=3 or 4, each possible subset among the 5 markers was evaluated and pooled as a single distribution. As expected, overall performance diminished with fewer markers used in the registration. As shown in FIGS. 9A and 9B, end-to-end performance for M=3 markers yielded median TRDₓ=4.7-6.9 mm and TRE_{φ}=3.7°-6.9° for N_{view}=1 or 2 with an unacceptably high rate of outliers. For M=4 markers, registration from N_{view}=1 was still unacceptably high (median TREₓ=4.2 mm and TRE_{φ}=3.8°), and improved considerably with N_{view}=2 (median TREₓ=2.2 mm and TRE_{φ}=2.8° with no outliers). For M=5, the end-to-end system performance with a single fluoroscopic view (N_{view}=1) gave median TREₓ=3.4 mm (1.9 mm IQR) and TRE_{φ}=2.7° (0.79° IQR), attributed primarily to out-of-plane error in marker localization with fluoroscopic images (FIGS. 7A and 7B). For M=5 markers and N_{view}=2, the end-to-end accuracy improved to median TREₓ=0.88 mm (0.16 mm IQR) and TRE_{φ}=2.0° (0.16° IQR).

### Videa-Fluoro and Video-CT Guidance

FIGS. 10A and 10B illustrate embodiments of each navigation mode and end-to-end system performance with 3D-2D registrations solved using either N_{view}=1 or 2 fluoroscopic images, taking the example of the AIIS-to-PSIS trajectory.

FIG. 10A shows augmented fluoroscopy guidance (Video-Fluoro mode) with commonly acquired fluoroscopic views. If a preoperative CT is available, the planned trajectory and acceptance corridors may also be overlaid (shown in green). Fluoroscopic views are overlaid with the registered drill axis trajectory in real-time according to the current pose of the drill—e.g., trajectories overlaid in FIG. 10A corresponding to N_{view}=1 or 2 (cyan or magenta, respectively). This mode of navigation allows the surgeon to adjust freehand drill trajectory in real-time to align with the planned trajectory, with the background anatomical scene providing important visual context. Since only the axis of the drill (cf., the “tip” of the K-wire) is conveyed, the trajectory overlay may be of primary benefit to the surgeon by helping to guide selection of the entry point and initial K-wire angulation, which are essential (and. challenging) to establishing a safe approach into the bone. Visualization in multiple augmented fluoroscopic views gives reliable 3D reckoning of the scene (normally done mentally, requiring years of training in understanding the complex morphology of the pelvis in projection views). To the extent that the presence of markers in the fluoroscopy image is visually distracting, they could easily be masked (e.g., by median filter), since the precise location of the wire and BB features was accurately determined in the marker detection step. Once the K-wire is advanced into the bone, of course, the K-wire tip is directly visible in the fluoroscopy image, and the overlays are confirmatory. This “Video-Fluoro” mode could be well suited to procedures for which no preoperative 3D imaging is available, as it establishes registration using only the fluoroscopic images collected in standard clinical workflow for fluoroscopically guided. procedures.

FIG. 10B shows augmented CT guidance (Video-CT mode) illustrated with multiplanar slices of the preoperative CT image. If a preoperative CT is available, the planned trajectory and acceptance corridors may also be overlaid (shown in green). The acceptance corridor and planned trajectory (green) are shown along with real-time rendering of the chill axis solved with N_{view}=1 (cyan) or N_{view}=2 (magenta). The drill axis trajectory is rendered in real-time according to the pose of the drill to aid the surgeon in determining whether the given trajectory conforms to the bone corridor. Such a 3D navigated view is common in neurosurgery and spine surgery via surgical trackers. The “Video-CT guidance” provided by the video drill system could help to bring the precision of 3D navigation to orthopaedic trauma surgery without the additional cost and workflow associated with tracking systems.

By updating “Video-Fluoro” or “Video-CT” visualization in real-time during freehand manipulation, the system could also help to reduce the amount of trial-and-error “fluoro hunting” in conventional workflow, thereby reducing time and radiation dose.

The trajectory shown in this example corresponds to K-wire delivery along the AIIS-to-PIIS bone corridor. The end-to-end system performance with a single fluoroscopic view (N_{view}=1) gave median TREₓ=3.4 mm (1.9 mm IQR) and TRE_{φ}=2.7° (0.79° IQR). For N_{view}=2, the end-to-end accuracy improved to median TREₓ=0.88 mm (0.16 mm IQR) and TRE_{φ}=2.0° (0.16° IQR).

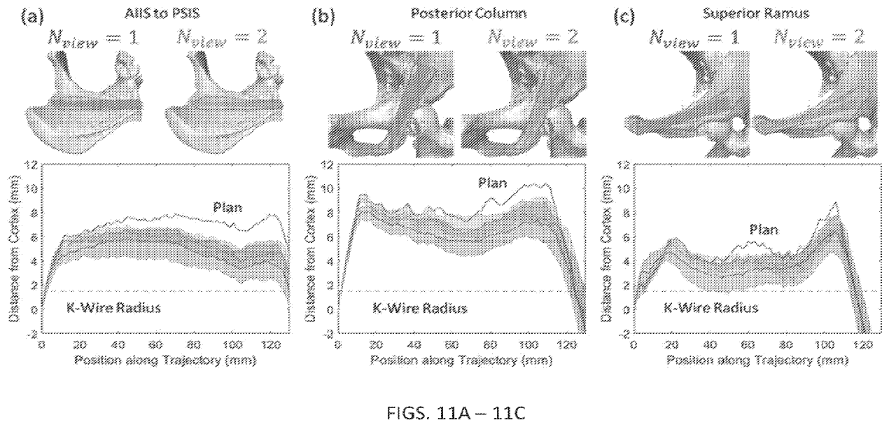

FIGS. 11A to 11C show conformance analysis and visualization of drill axis trajectories within the bone corridors of the (FIG. 11A) AIIS-to-PSIS, (FIG. 11B) posterior column, and (FIG. 11C) superior ramus for N_{view}=1 and N_{view}=2 fluoroscopic view registrations. The distributions reflect the distance between the predicted drill axis and the outer bone cortex as a function of distance along the drill axis. The horizontal dashed line represented the thickness of the K-wire used (1.5 mm radius), with values below this threshold indicating a breach of the bone cortex, in each case, a dispersion of simulated trajectories (computed by perturbing the planned trajectory according to a uniform distribution given by the median TREₓ and TRE_{ϕ}) are shown relative to a volumetric surface rendering of the pelvis phantom, with the acceptance corridor (region interior to bone cortex) shown in green and the range of simulated trajectories shown in blue and pink for N_{view}=1 and 2, respectively. Also shown in FIGS. 11A to 11C are plots of the distance between the registered drill axis and the bone cortex as a function of position along the trajectory. The distributions reflect the distance between the predicted drill axis and the outer bone cortex as a function of distance along the drill axis. Negative distance corresponds to a breach of the bone cortex, and each plot also shows a lower threshold (clashed line) representing the radius of a typical K-wire (1.5 mm), such that trajectories above the threshold are fully within the bone corridor, and values below this threshold indicating a breach of the bone cortex. For N_{view}=1, the average distance to cortex was 4.4 mm for AIIS-to-PSIS, 5.6 mm for PC, and 2.5 mm for SR—each without breach of the cortex (except minor impingement—˜0.1 mm—in a particularly narrow region of the central SR, which would likely correspond to a glancing incidence of the K-wire). For N_{view}=2, the average distance to cortex was 5.1 mm for AIIS-to-PSIS, 6.4 mm for PC, and 2.9 mm for SR—all well within the acceptance corridor.

### Experimental Evaluation: Cadaver Study Example

An embodiment of the proposed method was further tested in pre-clinical cadaver experiments. Geometric accuracy of the drill axis registration was evaluated with respect to common trajectories in pelvic trauma surgery [anterior inferior iliac spine (AIIS), superior ramus (SR), and posterior column (PC)]. A preoperative CT of the cadaver was obtained (Aquilion Precision CT, Canon) for automatic planning of common pelvic K-wire trajectories using atlas-based planning methods.^{39,40} Acceptance volumes within bone corridors were created for each trajectory and used to evaluate drill axis conformance within pelvic bone corridors.

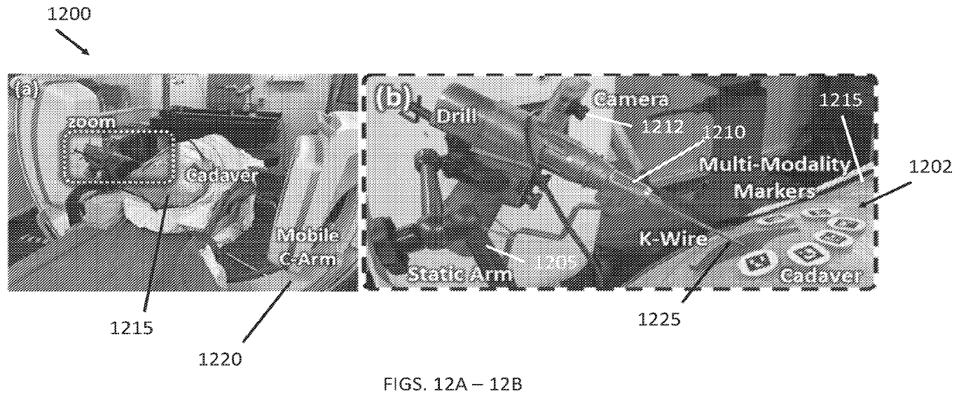

FIGS. 12A and 12B illustrate an experimental setup 1200 with a second-generation embodiment of the video-on-drill system for pre-clinical evaluation. FIG. 12A is a photograph of the cadaver setup. FIG. 12B is a magnified view showing components of the video-on-drill system in pre-clinical studies. Six multimodal markers 1202 were placed about each entry site to register and align the drill axis to the planned trajectory. Upon alignment, a static arm 1205 positioned the drill 1210 and camera 1212 at a distance of ˜20-25 cm from the cadaver 1215 to emulate realistic surgical drill positioning. An isocentric mobile C-arm 1220 (Cios Spin, Siemens) was used for fluoroscopic imaging (for 3D-2D registration) and CBCT (for truth definition of K-wire 1225 drill axis). System registration accuracy was evaluated using the same methods as the phantom study, reporting performance in terms of target registration error (TRE), conformance to clinically relevant pelvic bone K-wire corridors, and runtime.

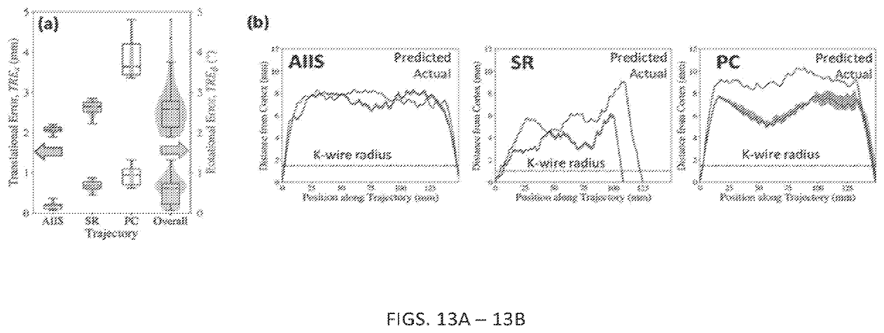

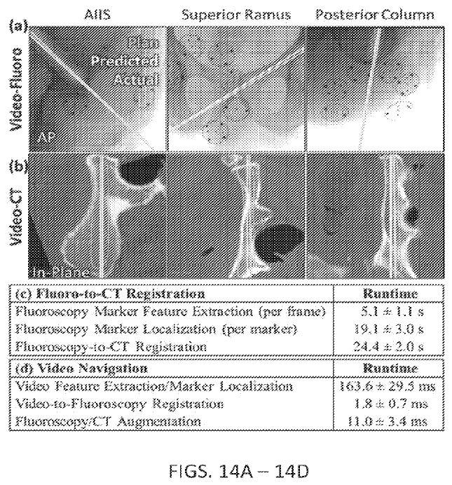

System registration accuracy is summarized in FIGS. 13A and 13B. FIG. 13A illustrates end-to-end drill axis registration error in terms of translational error at the entry point of the bone corridor (TRE_{X}) and rotational error (TRE_{φ}). Median TRE_{φ}for the AIIS, SR, and PC trajectories was 0.19° (0.12° IQR), 0.68° (0.16° IQR), and 0.95° (0.40° IQR), respectively. Median TREₓ for the AIIS, SR, and PC trajectories was 2.07 mm (0.10 mm IQR), 2.65 mm (0.24 mm IQR), and 3.64 mm (0.75 mm IQR), respectively. Overall, the system demonstrated median TRE_{φ}and TREₓ of 0.62° (0.51° IQR) and 2.6 mm (0.65 mm IQR), respectively.

FIG. 13B illustrates conformance of drill axis trajectories within the bone corridors of the AIIHS-to-PSIS, superior ramus (SR), and posterior column (PC). The dashed line represents the K-wire radius, below which corresponds to breach of the bone cortex. The average distance to cortex was 6.8 turn for AIIS-to-PSIS, 3.4 mm for SR, and 6.1 mm for PC—all well within the acceptance corridor.

FIGS. 14A and 14B illustrate visualization and guidance in (FIG. 14A) Video-Fluoro and (FIG. 14B) Video-CT navigation modes across multiple trajectories. In Video-Fluoro mode (FIG. 14A), the registered drill axis trajectory is projected in real-time relative to the planned trajectory (green). In Video-CT mode (FIG. 14B), 3D navigation is presented relative to preoperative CT, bringing the precision, accuracy, and safety of 3D navigation to a form that may be compatible with the steep cost and workflow requirements of orthopaedic trauma surgery.

The computational runtime of system is summarized in FIG. 14C for fluoroscopy/CT registration computed once (or each time the system must be re-registered to update after major deformation) and in FIG. 14D for video Navigation computed during freehand drill positioning. The total runtime for fluoroscopy/CT registration was 2.5 min total, noting that this step is normally performed only once. Total runtime for video guidance was 176.4±29.7 ms, giving a real-time update rate of ˜6 frames per second.

### Multimodal Fiducial Markers

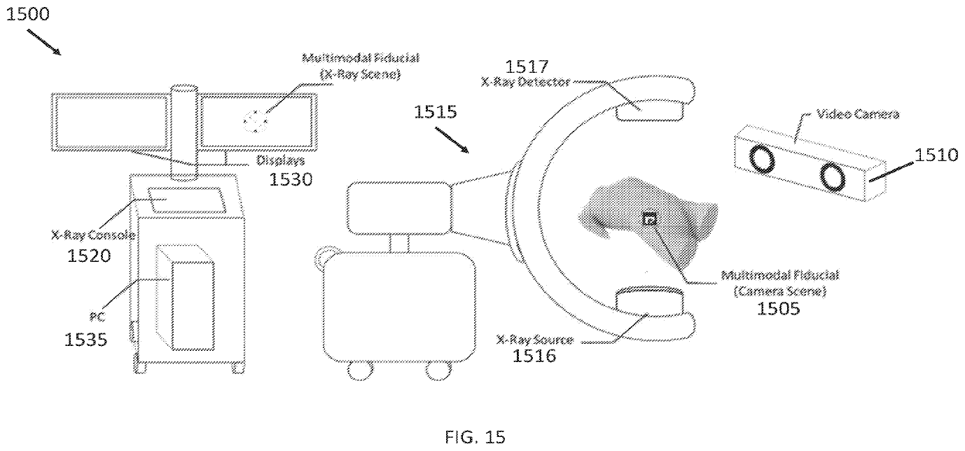

As discussed above, in some embodiments, multimodal fiducial markers visible to both a video camera and an imaging system (e.g., x-ray, cone-beam CT, MRF, etc.) are used to compute the geometric relationship between the camera and x-ray coordinate systems, allowing real-time, image-based surgical tracking in intraoperative x-ray images (as well as preoperative 3D images via 3D-2D registration³⁴). An example of an image-guided navigation system 1500 using multimodal fiducials 1505 to co-register scenes from a camera 1510 and x-ray system 1515 (including an x-ray source 1516 and an x-ray detector 1517) is schematically illustrated in FIG. 15. The system 1500 includes an x-ray console 1520 with one or more displays 1530 to display the x-ray scene. The x-ray console 1520 and the video camera 1510 are connected to a computer 1535 in some embodiments, to drive the displays and show one or both of the x-ray scenes and video scene separately on the displays 1530 or overlaid on each other on the displays 1530.

“Paired-Point” Markers. Some embodiments employ “paired-point” multimodal markers. These markers are named as such since they can be uniquely identified in both video and x-ray images. The markers are rigid, contain corresponding feature points detectable in video and x-ray images, and are matched prior to point-based registration.

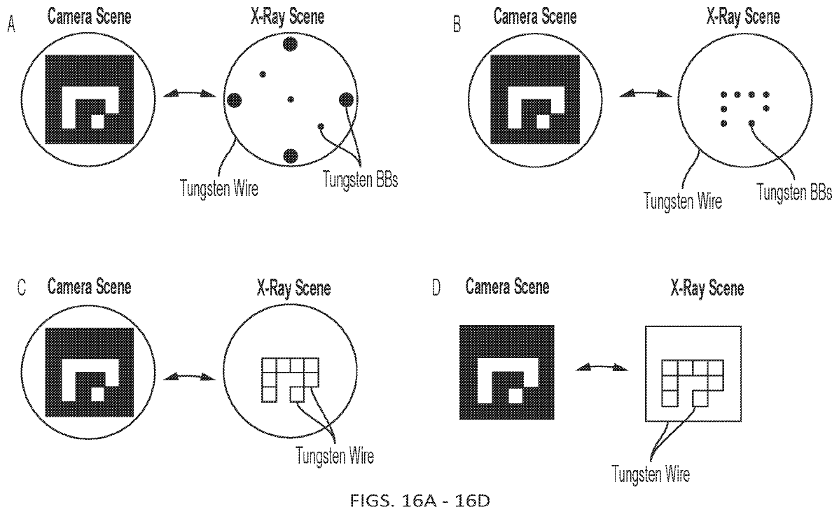

FIGS. 16A-16D illustrate multiple embodiments of “paired-point” multimodal markers. Optical features can be realized through various vision-based fiducial systems reported in the prior art (e.g. ArUco⁴¹) and are automatically detected, uniquely identified, and localized with a calibrated monocular/stereoscopic camera. Some embodiments for the markers in the x-ray scene make use of radio-opaque materials (e.g. tungsten, copper). FIG. 16A illustrates radio-opaque marker features such as a central ball bearing (BB) and a unique arrangement of outer BBs differing in size and location to identify each marker in x-ray images. To aid feature extraction in x-ray images, BBs are constrained to be collinear with at least two other BBs. Each marker is encircled by a tungsten wire to create a periphery that assists in marker detection and pose estimation. FIGS. 16B-16D illustrate alternative embodiments using radio-opaque materials that mimic the encoding schemes of the corresponding optical features, using the same underlying feature extraction and pose estimation methods. Specifically, FIG. 16B and 16C show a center of square tag and center of radio-opaque circle. FIG. 16D shows a center of square tag and center of radio-opaque square. Embodiments may be realized in many forms or shapes (e.g. circle, square, or other shapes).

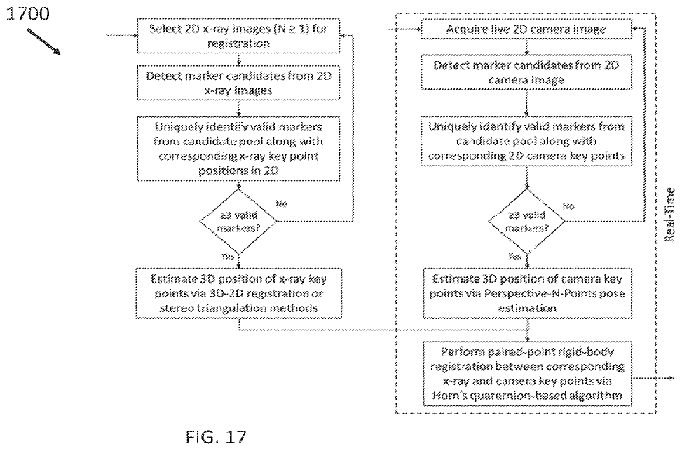

FIG. 17 depicts a 2D-3D registration process 1700 for some embodiments using “paired-point” markers with the following primary stages:

Markers (at least 3) are placed about the surgical field at the time of surgery.

X-ray images (one or more) are acquired with the markers in the field-of-view. Image processing algorithms automatically identify valid markers, determine marker identity, and extract key-point features. Marker correspondence between x-ray images is performed via direct identity matching.

If at least 3 markers are present across all x-ray images, the 3D position of x-ray key-point features is determined via automatic pose estimation techniques (e.g. 3D-2D registration, stereo triangulation)

Real-time video images of the markers are acquired, and automatic image processing algorithms uniquely identify corresponding markers and key-point features. The 3D position of video-based key-point features is determined via automatic pose estimate techniques (e.g. Perspective-N-Points algorithm).

If at least 3 corresponding markers are found between x-ray and video images, point-based registration methods (e.g. Horn's method) are used to register the video and x-ray scenes. After this stage, surgical devices are tracked and overlaid on x-ray images in real-time. Note that in some embodiments it is not required that all of the markers that are visible in both modalities are used to register the scenes. Other markers could be visible in both images, but which are unused for registration of the video and x-ray scenes.

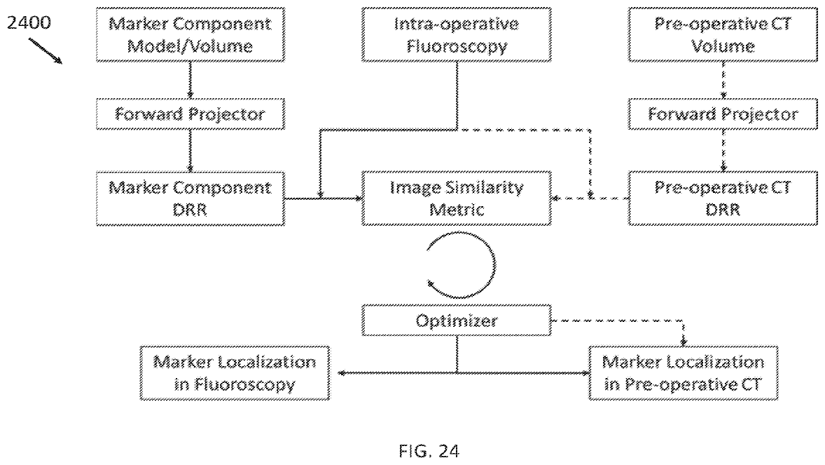

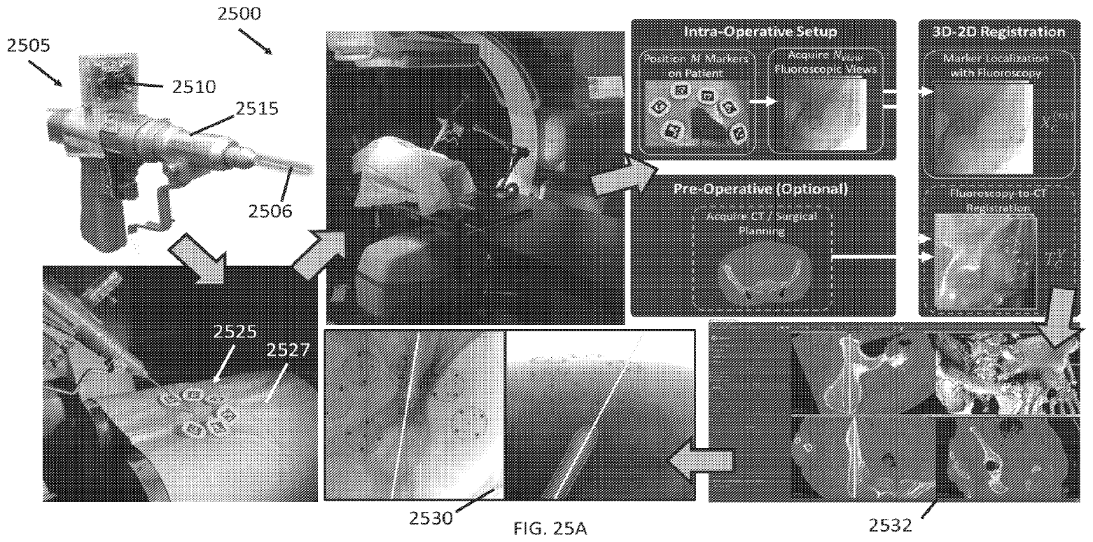

Registration with preoperative images is performed using an automatic 3D-2D image registration algorithm (e.g., as described below with reference to FIG. 24). In some embodiments, 3D-2D image registration does not require the presence of markers during 3D image acquisition. However, if markers are already present within the preoperative 3D image, the registration is established using the locations of the markers in both video and 3D-image, as per the prior art. After this stage, surgical devices are tracked and overlaid on 3D images in real-time.

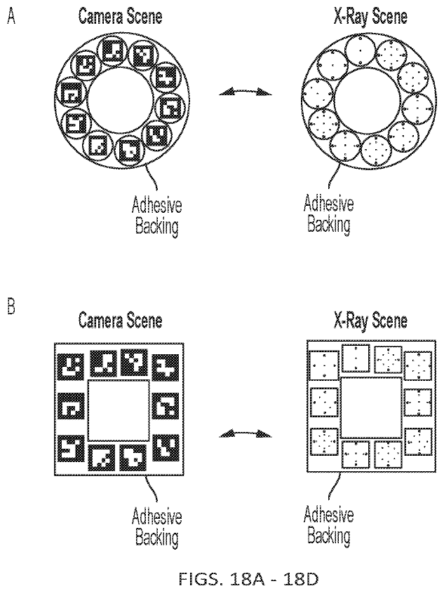

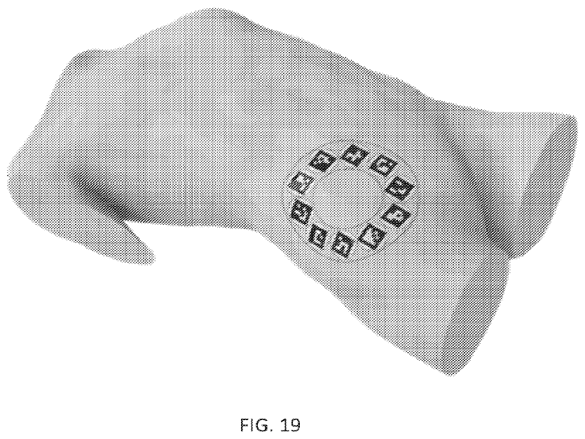

FIGS. 18A and 18B illustrate embodiments of “paired-point” marker arrangements that are compatible with clinical workflows. Arrangements take on multiple forms a circular arrangement as in FIG. 18A, or a rectangular arrangement as in FIG. 18B) with respect to the choice of “stray” multimodal marker used to represent point-based features. Some embodiments utilize a semi-flexible, adhesive-backed frame bearing the rigid. markers about an opening that is to be placed onto the surgical site (e.g., patient's surface), as shown in FIG. 19. The arrangement is robust to partial occlusion in video images since only a subset of markers are needed for registration and are easily readjusted during the procedure (cf. individual marker placement and readjustment). Other embodiments use an adhesive backing material in the form of an antimicrobial drape routinely applied in various clinical procedures (e.g. 3M Ioban)—embedded with “paired-point” markers arranged in a manner appropriate for the given procedure (e.g. circular, square). Such embodiments potentially realize image guided navigation with little to no impact on existing workflows.

“Point-Cloud” Markers. Some embodiments utilize “point-cloud” multimodal markers. These markers contain corresponding features points extracted in both video and x-ray images but are not uniquely identified and directly matched as in the “paired-point” approach. Instead a point cloud is generated in the video and x-ray space and conventional surface-matching algorithms are used to solve the registration.

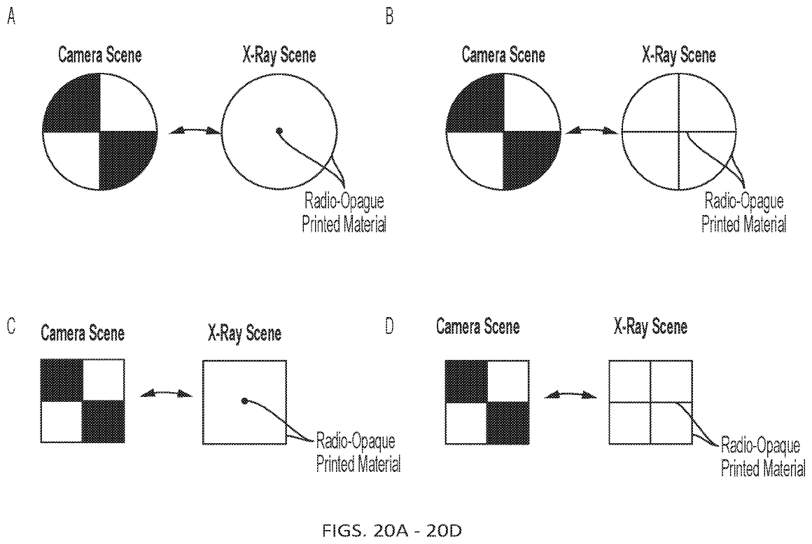

FIGS. 20A to 20D illustrate sample embodiments of point-cloud based markers. Optical features are realized through the same vision-based fiducial systems mentioned above or by conventional checkerboard-based patterns since only point-based feature correspondences are used (c.f. unique marker encodings). Embodiments in the x-ray scene include simple line and/or point-based patterns created using radio-opaque materials to represent relevant marker features with minimal disruption to the x-ray scene.

Embodiments are realized in many forms or shapes (e.g. circle, square, etc.). For example, FIGS. 20A and 20C illustrate embodiments of point-cloud based marker with a central corner and radio-opaque dot. FIGS. 20B and 20D illustrate embodiments of point-cloud based marker with central corner and radio-opaque line intersection.

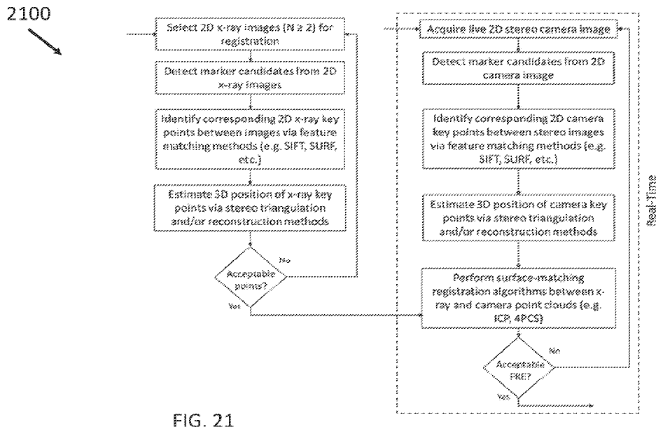

FIG. 21 depicts a registration process 2100 for some embodiments using “point-cloud” markers with the following primary stages:

Markers (at least 3) are placed about the surgical field at the time of surgery

X-ray images (2 or more) are acquired with the markers in the field-of-view. image processing algorithms automatically identify valid markers and extract key-point features. Marker correspondence between x-ray images is performed via off-the-shelf feature matching techniques (e.g., SIFT, SURF).

If an acceptable number of key-points are found, the 3D position of x-ray key-point features is then determined via automatic pose estimation techniques (e.g. 3D-2D registration, stereo triangulation).

Real-time stereo video images of the markers are acquired, and automatic image processing algorithms detect key-point features. The 3D position of video-based key-point features is determined via automatic 3D reconstruction techniques (e.g. stereo triangulation, structure from motion).

If a sufficient number of points are used to create x-ray and video point-clouds, off-the-shelf surface-matching registration methods (e.g. iterative closest point) are used to register the video and x-ray scenes. Following registration, the fiducial registration error (FRE) is checked and if within an acceptable level, the registration is treated as successful. After this stage, surgical devices are tracked and overlaid on x-ray images in real-time.

Registration with preoperative images can be performed using an automatic 3D-2D image registration algorithm (e.g., as described below with reference to FIG. 24). 3D-2D image registration does not require the presence of markers during 3D image acquisition. However, if markers are already present within the preoperative 3D image, the registration can also be established using the locations of the markers in both video and 3D-image, as per the prior art. After this stage, surgical devices can be tracked and overlaid on 3D images in real-time.

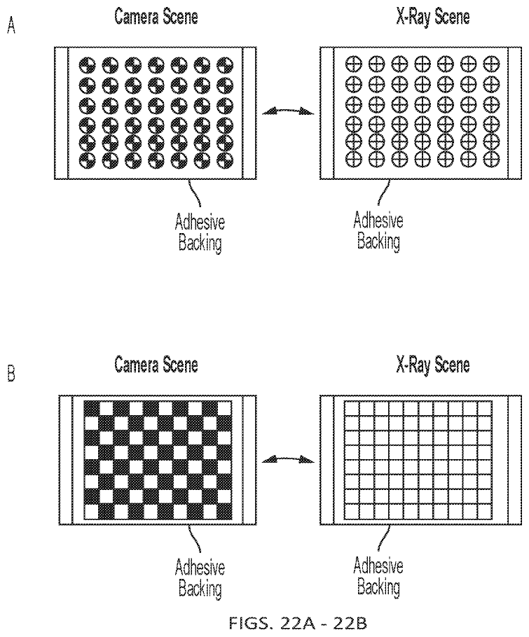

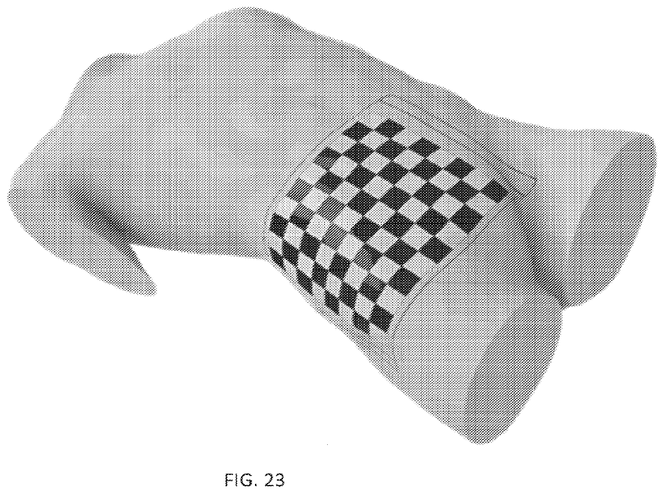

FIGS. 22A and 22B illustrate sample embodiments of “point-cloud” marker arrangements that are compatible with clinical workflows. Arrangements take on multiple forms, such as circular (FIG. 22A) and rectangular (FIG. 22B) with respect to the choice of “stray” multimodal marker used to represent point-based features. Some embodiments (e.g. as illustrated in FIG. 23, for a patient surface) are realized as a chessboard pattern printed onto a flexible, adhesive-backed surgical drape, containing corresponding radio-opaque features in the form of a grid (corners of the grid pattern realize key-point features used for point-cloud registration). The registration is robust to surface deformations in the drape (e.g. due to incision, drape lift) since such deformations are consistent between video and x-ray images. Furthermore, the registration is robust in some embodiments to poor detection and outlier points, given a sufficiently large point cloud, Since adhesive-backed drapes are routinely , employed in clinical procedures (e.g. 3M Ioban antimicrobial drape), these embodiments have the potential to establish image guided navigation with little to no impact on existing workflows.

FIG. 24 illustrates a flowchart 2400 for 3D-2D registration of multimodal markers. Solid lines represent the flow for registration of markers to intraoperative fluoroscopy via a known-component model of the marker. Dashed lines represent the flow for registration of preoperative images to intraoperative fluoroscopy. Combination of both allows for localization of markers with preoperative 3D images.

Marker Design, FIG. 3B above shows an exploded view of multimodal markers used in some embodiments to relate the drill camera and C-arm frames via optical and radio-opaque features, Optical features are presented by Artico marker tags consisting of a 6×6 inner matrix of bits to uniquely identify each marker. Each marker contained a central ball bearing (BB) (e.g., tungsten, 2.0 mm) and a unique arrangement of outer BBs differing in size (e.g., 2.0 or 3.5 mm) and location to identify each marker in fluoroscopic images. To aid feature extraction in fluoroscopy, BBs were constrained in some embodiments to be collinear with at least two other BBs. Each marker was encircled in some embodiments by a tungsten wire (e.g., 0.8 mm) to create a periphery (e.g., 48 mm) that assisted in marker detection (by Bough transform) and pose estimation (circle-to-ellipse perspective relationship),

In some embodiments, the marker base was 3D-printed (Vero PureWhite, Connex-3 Objet 260, Stratasys, Eden Prairie, MN, LISA) with pockets to hold the BBs, a peripheral groove to hold the wire, and a 30×30 mm² square recess (˜1.8 mm deep) in which the ArUco tag was placed such that its center coincided with the central BB. The initial design had a footprint of 50 mm and could generate up to 48 unique markers, with other embodiments having more compact designs.

Marker Detection. The detection of ArUco tags in video was based in some embodiments on open-source tools available in OpenCV. The algorithm of some embodiments first performs adaptive thresholding and contour detection of grayscale images to isolate candidate regions for multiple tags. The inner area of each candidate is then analyzed by correcting the tag perspective to a square region and then binarizing the resulting region to a regularly spaced grid upon which marker identification can be performed.

In some embodiments, marker detection in the fluoroscopic scene was performed first by ellipse detection (using the peripheral wire) to coarsely identify individual marker positions. A Canny edge filter was followed by morphological closing to yield binafized elliptical contours and filter out smaller Objects (e.g., BBs). A Hough-based ellipse detector returned elliptical fits ordered by accumulator score, which was cutoff according to the known number of markers (M).

The inner region within each ellipse was then analyzed to determine the arrangement of BBs and the corresponding unique marker ID. A morphological top-hat filter isolated marker features from surrounding anatomy, and the ellipse was removed by morphological opening to isolate the BB features. Hough-based circle detection was then used to identify the position and radius of BB locations within each marker. To eliminate false positives, candidate BBs were filtered based on the known range of BB radii. Detections within a certain proximity to each other were also filtered based on the known marker designs, and collinearity was enforced to remove any remaining false positives. The resulting BB detections were then hierarchically clustered in two groups according to size, and markers were uniquely identified according to a lookup table.

### Generality and Alternative Embodiments of the Proposed Solution

The embodiments discussed above realize surgical guidance using the video-on-drill concept in the example context of K-wire insertions in orthopaedic-trauma surgery. Embodiments can be generalized for tracking a variety of surgical instrumentation (e.g. drill bit, screws, nails, biopsy needles, etc.). Embodiments of the system can also be envisioned using multiple combinations of stereo/mono video cameras, C-arm/O-arm/portable x-ray intraoperative imaging devices, and CT/MRI/cone-beam CT (CBCT) 3D images. To achieve navigation, a video camera can in principle be mounted on other locations (e.g., handheld instrument, overhead, or tablet computer) as long as it can observe instruments with patient surface markers. Other embodiments replace the video camera altogether, using an alternative tracking modality (e.g. infrared, electromagnetic) with corresponding multimodal markers (e.g. active infrared LEDs, electromagnetic sensor coils) for registration with fluoroscopic images.

A potential embodiment can be envisioned in which naturally occurring features are present in both the camera and fluoroscopic images replace the requirement for surface markers positioned on the patient. For example, naturally occurring features such as the surface of bony anatomy exposed at the surgical site (e.g. features of the pelvic ring) and/or instrumentation part of the surgical setup (e.g. surgical retractor) would provide the basis for video-to-fluoroscopy registration. A stereoscopic video camera detects features in the 3D space of the patient (alternatively, determined by 3D structure-from-motion approach using a monocular video camera), and the x-ray fluoroscopy system detects such features in 2 or more fluoroscopy images to localize them in the 3D space of the x-ray imaging system. The two sets of 3D features are then co-registered to establish registration between the camera (mounted on the drill) and the x-ray imaging system. Such an embodiment would automatically detect corresponding features in video and x-ray images without relying upon known feature configurations in the markers.

The examples above augment the real-time location of the instrument onto x-ray/CT images, recognizing that alternative embodiments similarly augment anatomy from x-ray/CT onto the camera images. This would provide an augmented reality display of the anatomy underneath the area visible to the video camera. If the intended trajectory is defined in the preoperative 3D image, it can be overlaid on video images such that the surgeon can align the trajectory of the drill to what was defined in the preoperative image.

Other embodiments are applicable to other medical procedures outside ortho-trauma surgery in which the physician needs to intraoperatively estimate and verify the 3D pose of a medical instrument relative to surrounding anatomy. Examples include various needle injection procedures, such as spinal pain management, biopsy procedures, or ablation procedures.

Embodiments outside the medical domain are similarly applicable. For example, potential embodiments include visual servo-ing and vision-based robotic guidance where the 3D pose of an instrument needs to be tracked. In other words, the video-guided instrument is not limited to medical instruments in some embodiments.

The term “computer” is intended to have a broad meaning that may be used in computing devices such as, e.g., but not limited to, standalone or client or server devices. The computer may be, e.g., (but not limited to) a personal computer (PC) system running an operating system such as, e.g., (but not limited to) MICROSOFT® WINDOWS® NT/98/2000/XP/Vista/Windows 7/8/etc. available from MICROSOFT® Corporation of Redmond, Wash., U.S.A. or an Apple computer executing MAC® OS from Apple® of Cupertino, Calif., U.S.A. However, the invention is not limited to these platforms. Instead, the invention may be implemented on any appropriate computer system running any appropriate operating system. In one illustrative embodiment, the present invention may be implemented on a computer system operating as discussed herein. The computer system may include, e.g., but is not limited to, a main memory, random access memory (RAM), and a secondary memory, etc. Main memory, random access memory (RAM), and a secondary memory, etc., may be a computer-readable medium that may be configured to store instructions configured to implement one or more embodiments and may comprise a random-access memory (RAM) that may include RAM devices, such as Dynamic RAM (DRAM) devices, flash memory devices, Static RAM (SRAM) devices, etc.

The secondary memory may include, for example, (but is not limited to) a hard disk drive and/or a removable storage drive, representing a floppy diskette drive, a magnetic tape drive, an optical disk drive, a compact disk drive CD-ROM, flash memory, etc. The removable storage drive may, e.g., but is not limited to, read from and/or write to a removable storage unit in a well-known manner. The removable storage unit, also called a program storage device or a computer program product, may represent, e.g., but is not limited to, a floppy disk, magnetic tape, optical disk, compact disk, etc. which may be read from and written to the removable storage drive. As will be appreciated, the removable storage unit may include a computer usable storage medium having stored therein computer software and/or data.

In alternative illustrative embodiments, the secondary memory may include other similar devices for al lowing computer programs or other instructions to be loaded into the computer system. Such devices may include, for example, a removable storage unit and an interface. Examples of such may include a program cartridge and cartridge interface (such as, e.g., but not limited to, those found in video game devices), a removable memory chip (such as, e.g., but not limited to, an erasable programmable read only memory (EPROM), or programmable read only memory (PROM) and associated socket, and other removable storage units and interfaces, which may allow software and data to be transferred from the removable storage unit to the computer system.

The computer may also include an input device may include any mechanism or combination of mechanisms that may permit information to be input into the computer system from, e.g., a user. The input device may include logic configured to receive information for the computer system from, e.g. a user. Examples of the input device may include, e.g., but not limited to, a mouse, pen-based pointing device, or other pointing device such as a digitizer, a touch sensitive display device, and/or a keyboard or other data entry device (none of which are labeled). Other input devices may include, e.g., but not limited to, a biometric input device, a video source, an audio source, a microphone, a web cam, a video camera, and/or another camera. The input device may communicate with a processor either wired or wirelessly.

The computer may also include output devices which may include any mechanism or combination of mechanisms that may output information from a computer system. An output device may include logic configured to output information from the computer system. Embodiments of output device may include, e.g., but not limited to, display, and display interface, including displays, printers, speakers, cathode ray tubes (CRTs), plasma displays, light-emitting diode (LED) displays, liquid crystal displays (LCDs), printers, vacuum florescent displays (VFDs), surface-conduction electron-emitter displays (SEDs), field emission displays (FEDs), etc. The computer may include input/output (I/O) devices such as, e.g., (but not limited to) communications interface, cable and communications path, etc. These devices may include, e.g., but are not limited to, a network interface card, and/or modems. The output device may communicate with processor either wired or wirelessly. A communications interface may allow software and data to be transferred between the computer system and external devices.

The term “data processor” is intended to have a broad meaning that includes one or more processors, such as, e.g., but not limited to, that are connected to a communication infrastructure (e.g., but not limited to, a communications bus, cross-over bar, interconnect, or network, etc.). The term data processor may include any type of processor, microprocessor and/or processing logic that may interpret and execute instructions (e.g., for example, a field programmable gate array (FPGA)). The data processor may comprise a single device (e.g., for example, a single core) and/or a group of devices (e.g., multi-core). The data processor may include logic configured to execute computer-executable instructions configured to implement one or more embodiments. The instructions may reside in main memory or secondary memory. The data processor may also include multiple independent cores, such as a dual-core processor or a multi-core processor. The data processors may also include one or more graphics processing units (GPU) which may be in the form of a dedicated graphics card, an integrated graphics solution, and/or a hybrid graphics solution. Various illustrative software embodiments may be described in terms of this illustrative computer system. After reading this description, it will become apparent to a person skilled in the relevant art(s) how to implement the invention using other computer systems and/or architectures.

The term “data storage device” is intended to have a broad meaning that includes removable storage drive, a hard disk installed in hard disk drive, flash memories, removable discs, non-removable discs, etc. In addition, it should be noted that various electromagnetic radiation, such as wireless communication, electrical communication carried over an electrically conductive wire (e.g., but not limited to twisted pair, CAT5, etc.) or an optical medium (e.g., but not limited to, optical fiber) and the like may be encoded to carry computer-executable instructions and/or computer data that embodiments of the invention on e.g., a communication network. These computer program products may provide software to the computer system. It should be noted that a computer-readable medium that comprises computer-executable instructions for execution in a processor may be configured to store various embodiments of the present invention.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

### Further Embodiments

The following is an example of a further embodiment within the general concepts of this invention. The general concepts are not limited to this and/or the other specific embodiments that were described in detail to facilitate an explanation of some concepts of the current invention. The scope of the invention is defined by the claims.

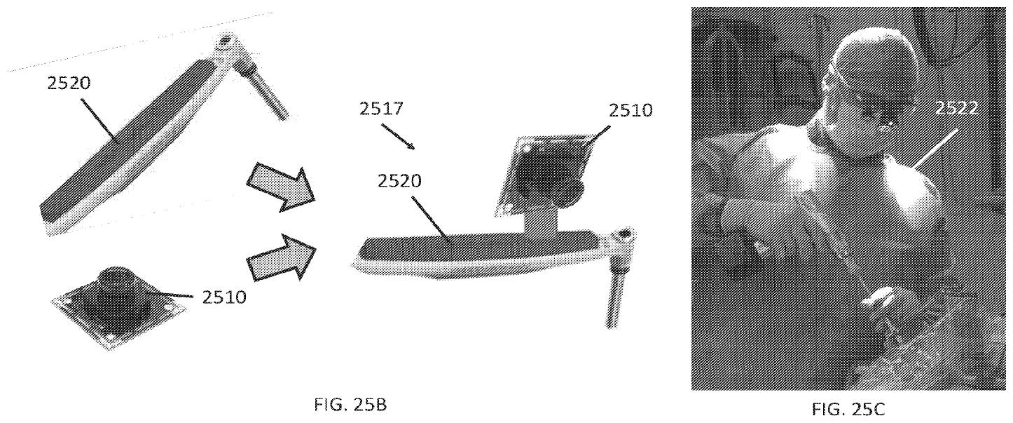

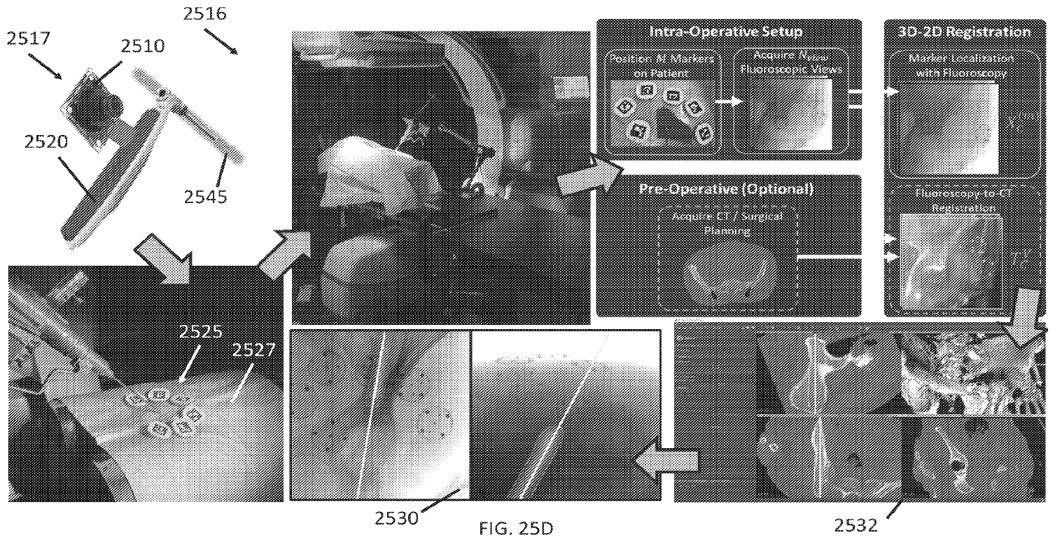

FIG. 25A illustrates a workflow 2500 for an embodiment of a video-guided drill, i.e. a video drill 2505. This embodiment is a video camera 2510 on-board a surgical drill 2515. FIG. 25B illustrates another embodiment, for a video drill guide 2517, where the video camera 2510 is on-board a drill guide 2520 instead of the drill 2515. FIG. 25C illustrates how a surgeon 2522 uses a drill guide 2520. FIG. 25D illustrates a workflow 2516 for the embodiment of the video drill guide 2517. Both embodiments of a video drill 2505 and a video drill guide 2517 may be implemented using a system such as the system described above with reference to FIG. 1A and FIG. 5A. The video drill guide 2517 may use the exact same process, algorithms, and workflow as the video drill 2505.

In FIG. 25A, for the embodiment of the video drill 2505, the drill axis 2506 is defined relative to the video scene from the video camera 2510. Markers 2525 are positioned on a patient (represented in this example by a phantom 2527) and are detected in the video from the video camera 2510, using a technique such as that described above with reference to FIGS. 2A to 2D. A registration algorithm, such as that described above with reference to FIG. 4, is used to relate the video scene (drill axis 2506) to x-ray fluoroscopy and/or to preoperative CT. This then allows for real-time guidance, by visualization of the drill axis 2506, overlaid onto fluoroscopy images 2530 and/or CT images 2532.

FIG. 25B illustrates a video-guided drill guide, i.e., a video drill guide 2517, of some embodiments. The video camera 2510 is here attached to the drill guide 2520. FIG. 25C shows how a surgeon 2522 uses a drill guide: First, the surgeon 2522 aligns the drill guide 2520 with their off-hand (e.g., their left hand, if they are right-handed). The surgeon 2522 uses the drill guide 2520 to get a good purchase of bone at the entry point, and align the desired trajectory (as visualized in fluoroscopy). The drill 2515 and the K-wire are then brought into the drill guide 2520, to drill the trajectory with their primary hand (e.g. their right hand, if right-handed). The surgeon 2522 uses the video drill guide 2517 in exactly the same manner as a standard drill guide 2520, the only difference with the video drill being that the video camera 2510 is affixed to the drill guide 2520 instead of the drill 2515.

In FIG. 25D, for the embodiment of the video drill guide 2517, the drill guide axis 2545 is defined relative to the video scene from the video camera 2510. Markers 2525 are positioned on a patient (represented in this example by a phantom 2527) and are detected in the video from the video camera 2510, using a technique such as that described above with reference to FIG. 2. A registration algorithm, such as that described above with reference to FIG. 4, is used to relate the video scene (drill guide axis 2545) to x-ray fluoroscopy and, or to preoperative CT. This then allows for real-time guidance, by visualization of the drill guide axis 2545, overlaid onto fluoroscopy images 2530 and/or CT images 2532.

### REFERENCES

1. Burge R, Dawson-Hughes B, Solomon D H, Wong I B, King A, Tosteson A. Incidence and economic burden of osteoporosis-related fractures in the United States, 2005-2025. J Bone Miner Res. 2007; 22(3):465-475. doi: 10.1359/jbmr.061113
2. Buller L T, Best M J, Quinnan S M. A Nationwide Analysis of Pelvic Ring Fractures. Geriatr Orthop Surg Rehabil, 2016; 7(1):9-17. doi:10,1177/2151458515616250
3. Balogh Z, ling K L, Mackay et al. The epidemiology of pelvic ring fractures: A population-based study. J Trauma—Inj infect Crit Care. 2007; 63(5):1066-1072. doi:10.1097/TA.0b013e3181589fa4
4. Davis D D, Foris L A, Kane S M, Waseem M. Pelvic Fracture, StatPearls Publishing, 2020. Accessed Jun. 22, 2020, http://www.ncbi.nlm.nih.gov/pubmed/28613485
5. Poole G V., Ward E F, Griswold J A, Muakkassa F F, Hsu H S H. Complications of pelvic fractures from blunt trauma. In: American Surgeon. Vol 58.; 1992:225-231. Accessed Jun. 22, 2020. https://europepmc.org/article/med/1586080
6. Yoshihara H, Yoneoka D. Demographic epidemiology of unstable pelvic fracture in the United States from 2000 to 2009: Trends and in-hospital mortality. J Trauma Acute Care Surg. 2014; 76(2):380-385. doi:10.1097/TA.0b013e3182ab0cde
7. Van Den Bosch E W, Van Der Kleyn R, Hogervorst M, Van Vugt A B. Functional outcome of internal fixation for pelvic ring fractures. J Trauma—Inj Infect Crit Care. 1999; 47(21:365-371. doi: 10.1097/00005373-199908000-00026
8. Burgess A R, Eastridge B J, Young J W R, et al. Pelvic ring disruptions: Effective classification system and treatment protocols. J Trauma—Inj Infect Crit Care. 1990; 30(7):848-856. doi: 10.1097/00005373-199007000-00015
9. Gao H, Luo C F, Zhang C Q, Zeng B F. Minimally invasive fluoro-navigation screw fixation for the treatment of pelvic ring injuries. Surg Innov. 2011; 18(3):279-284, doi:10.1177/1553350611399587
10. Gras F, Marintschev I, Wilharm A, Klos K, Mückley T, Hofmann G O. 2D-fluoroscopic navigated percutaneous screw fixation of pelvic ring injuries—A case series. BMC Musculoskelet Discord. 2010; 11. doi: 10.1186/1471-2474-11-153
11. Hilgert R E, Finn J, Egbers H J. Technik der perkutanen SI-verschraubung mit unterstützung Burch konventionellen C-Bogen. Unfallchirurg. 2005; 108(11):954-960. doi:10.1007/s00113-005-0967-3
12. Helm P A, Teichman R, Hartmann S L, Simon D. Spinal Navigation and Imaging: History, Trends, and Future. IEEE Trans Med Imaging. 2015; 34(8):1738-1746. doi:10.1109/TMI2015.2391200
13. Härtl R, Lam K S, Wang J, Korge A, Kandziora F. Audigé L. Worldwide survey on the use of navigation in spine surgery. World Neurosurg. 2013; 79(1):162-172. doi:10.1016/j.wneu.2012.03.011
14. Navab N, Bani-Kashemi A, Mitschke M. Merging visible and invisible: Two Camera-Augmented Mobile C-arm (CAMC) applications. In: Proceedings—2nd IEEE and ACM International Workshop on Augmented Reality, IWAR 1999. institute of Electrical and Electronics Engineers Inc.; 1999:134-141. doi:10.1109/IWAR.1999.803814
15. Traub J; Heibel T H, Dressei P, Heining S M, Graumann 11. ; Navab N. A Multi-view Opto-Xray Imaging System. In: Medical image Computing and Computer-Assisted Intervention—MICCAI 2007. Springer Berlin Heidelberg; 2007:18-25. doi:10.1007/978-3-540-75759-73
16. Navab N, Reining S M, Traub J. Camera augmented mobile C-arm (CAMC): Calibration, accuracy study, and clinical applications. IEEE Trans Med. Imaging. 2010;29(7):1412-1423. doi:10.1109/TMI.2009,2021.947
17. Reaungamornrat S, Otake Y, Uneri⋅A, et al. An on-board surgical tracking and video augmentation system for C-arm image guidance. Int J CARS. 2012; 7:647-665. doi:10.1007/s11548-012-0682-9
18. Yoo J, Schafer S, Uneri A, Otake Y, Khanna A J, Siewerdsen electromagnetic “tracker-in-Table” configuration for X-ray fluoroscopy and cone-beam CT-guided surgery. Int J Comput Assist Radiol Surg. 2013; 8(1):1-13. doi:10.1007/s11548-012-0744-z
19. Magaraggia J, Wei W, Weiten M, et al. Design and evaluation of a portable intra-operative unified-planning-and-guidance framework applied to distal radius fracture surgery. Int J CARS. 2017; 12:77-90. doi:10.1007/s11548-016-1432-1
20. Von Jako CR, Luk Y, Zur O, Gilboa P. A novel accurate minioptical tracking system for percutaneous needle placement. IEEE Trans Biomed Eng. 2013; 60(8):2222-2225. doi: 10, 1109/TBME.2013.2251883
21. Vetter S Y, Magaraggia J, Beisemann N, et al. Virtual guidance versus virtual implant planning system in the treatment of distal radius fractures. Int J Med Robot Comput Assist Surg. 2018; 14(5):e1945. doi:10.1002/rcs.1945
22. Hamming N M, Daly M J, Irish J C, Siewerdsen I H. Automatic image-to-world registration based on x-ray projections in cone-beam CT-guided interventions. Med Phys. 2009;36(5):1800-1812. doi:10.1118/1.3117609
23. Dang H, Otake Y, Schafer S, Stayman J W, Kleinszig G, Siewerdsen J H. Robust methods for automatic image-to-world registration in cone-beam CT interventional guidance. Med Phys. 2012; 39(10):6484-6498. doi:10.1118/1.4754589
24. Andress S, Johnson A, Unberath M, et al. On-the-fly augmented reality for orthopedic surgery using a multimodal fiducial. Med Imaging. 2018; 5(02):1 doi: 10.1117/1.jmi.5.2.021209
25. Basafa E, Holβbach M, Stoika P J Fast, intuitive, vision-based: Performance metrics for visual registration, instrument guidance, and image fusion. In: Lecture Notes in Computer Science (Including Subseries Lecture Notes in Artificial Intelligence and Lecture Notes in Bioinformatics). Vol 9958 LNCS. Springer Verlag; 2016:9-17. doi:10.1007/978-3-319-46472-5_2
26. Shahidi R, Bax M R, Maurer C R, et al. Implementation, calibration and accuracy testing of an image-enhanced endoscopy system. IEEE Trans Med imaging. 2002; 21(12):1524-1535. doi:10.1109/TMI2002.806597
27. Maurer C R, Michael Fitzpatrick J, Wang M Y, Galloway I R L, Maciunas R J Allen G S. Registration of head volume images using implantable fiducial markers IEEE Trans Med Imaging. 1997; 16(4):447-462. doi:10.1109/42.611354
28.Roessler K, Ungersboeck K, Dietrich W, et al. Frameless stereotactic guided neurosurgery: Clinical experience with an infrared based pointer device navigation system. Acta Neurochir (Wien). 1997; 139(6): 551-559, doi:10.1007/BF02750999
29. Grunert P, Espinosa J, Busert C, et al. Stereotactic biopsies guided by an optical navigation system: Technique and clinical experience. Minim Invasive Neurosurg. 2002; 45(1): 11-15, dot: 10.1055/s-2002-23576
30. Zhang Z. A flexible new technique for camera calibration. IEEE Trans Pattern Anal Mach Intell. 2000; 22(11):1330-1334. doi:10.1109/34.888718
31. Tsai R Y, A Versatile Camera Calibration Technique for High-Accuracy 3D Machine Vision Metrology Using Off-the-Shelf TV Cameras and Lenses. Vol 3.; 1987. doi:10.1109/JRA.1987.1087109
32. Vagdargi P, Uneri A, Sheth N, et al. Calibration and registration of a freehand video-guided surgical drill for orthopaedic trauma. In: Vol 11315. SPIE-Intl Soc Optical Eng; 2020:15. doi: 10.1.117/12.2550001
33. Uneri A, Stayman J W, De Silva T, et al. Known-component 3D-2D registration for image guidance and quality assurance in spine surgery pedicle screw placement. In: Medical Imaging 2015: Image-Guided Procedures, Robotic Interventions, and Modeling. Vol 9415. SPIE; 2015:94151F. doi:10.1117/12.2082210
34. Otake Y, Wang A S, Webster Stayman J, et al. Robust 3D-2D image registration: Application to spine interventions and vertebral labeling in the presence of anatomical deformation. Phys Med Biol. 2013; 58(23):8535-8553. doi:10.1088/0031-9155/58/23/8535
35. Otake Y, Schafer S, Stayman J W, et al. Automatic localization of vertebral levels in x-ray fluoroscopy using 3D-2D registration: A tool to reduce wrong-site surgery. Phys Med. Biol. 2012; 57(17):5485-5508. doi:10.1088/0031-9155/57/17/5485
36. Hansen N, Osterineier A. Completely derandomized self-adaptation in evolution strategies. Evol Comput. 2001; 9(2):159-195. doi: 10.1162/106365601750190398
37. Horn B K P. Closed-form solution of absolute orientation using unit quaternions. J Opt Soc Am A. 1987; 4(4):629. doi:10.1364/josaa.4.000629
38. De Silva T, tined A, Ketcha M D, et al. 3D-2D image registration for target localization in spine surgery: Investigation of similarity metrics providing robustness to content mismatch. Phys Med Biol. 2016; 61(8):3009-3025. doi:10.1088/0031-9155/61/8/3009
39. Goerres J, Uneri A, Jacobson M, et al. Planning, guidance, and quality assurance of pelvic screw placement using deformable image registration. Phys Med Biol. 2017; 62(23):9018-9038, doi:10.1088/1361-6560/aa954f
40. Han R, Linen A, De Silva T, et al. Atlas-based automatic planning and 3D-2D fluoroscopic guidance in pelvic trauma surgery. Phys Med Biol. 2019; 64:17. doi:10.1088/1361-6560/ab1456
41. Garrido-Jurado S, Muñoz-Salinas R, Madrid-Cuevas F J, Medina-Carnicer R. Generation of fiducial marker dictionaries using Mixed integer Linear Programming, Pattern Recognit. 2016; 51:481-491. doi:10.1016/j.patcog.2015.09.23
42. Torr P H S, Zisserrnan A. MLESAC: A new robust estimator with application to estimating image geometry. Comput Vis Image Underst. 2000; 78(1):138-156. doi:10.1006/cviu.1999.0832
43. Cho Y, Moseley D J, Siewerdsen J H, Jaffray D A. Accurate technique for complete geometric calibration of cone-beam computed tomography systems. Med Phys. 2005; 32(4):968-983. doi:10.1118/1.1869652
44. Han R, Uneri A, Wu P, et al. Multi-body registration for fracture reduction in orthopaedic trauma surgery. In: Fei B, Linte C A, eds. Medical Imaging 2020: Image-Guided Procedures, Robotic Interventions, and Modeling. Vol 11315, SPIE-Intl Soc Optical Eng; 2020:14. doi:10.1117/12.2549708
45. Han R., Uneri A, Vijayan R C, et al, A Multi-Body Image Registration Framework for Pelvic Fracture Reduction and Guidance in Orthopaedic Trauma Surgery. Med Image Anal.:(submitted, pending review).

We claim:

1. A system for surgical navigation, comprising:
an instrument configured for a medical procedure on a patient;
a camera attached to the instrument, wherein the instrument has a spatial position relative to the camera;
an x-ray system configured to acquire x-ray images of the patient during the medical procedure;
a plurality of fiducial markers positioned on the surface of the patient during the medical procedure, said fiducial markers being detectable by both the camera and the x-ray system, said fiducial markers comprising a radio-opaque material arranged as at least one of a line and a point; and
a computer configured to:
receive an optical image acquired by the camera;
receive an x-ray image acquired by the x-ray system;
identify a subset of the fiducial markers that are visible in the optical image and are also visible in the x-ray image;
determine, based on the optical image, a spatial position relative to the camera for each fiducial marker in the subset of fiducial markers;
determine, based on the x-ray image, a spatial position relative to the x-ray system for each fiducial marker in the subset of fiducial markers; and
determine, based on at least the spatial positions of the subset of fiducial markers relative to the camera and the spatial positions of the subset of fiducial markers relative to the x-ray system, a spatial position for the instrument relative to the x-ray system.

2. The system of claim 1, wherein the computer is further configured to display the spatial position of the instrument relative to the x-ray system as an overlay on the x-ray images.

3. The system of claim 1, wherein the computer is further configured to:
receive a computed tomography (CT) image acquired from a CT system prior to the medical procedure;
perform a registration of the x-ray image to the CT image;
based on the registration of the CT image to the x-ray image, display the spatial position of the instrument relative to the x-ray system as an overlay on the CT image.

4. The system of claim 3, wherein none of the fiducial markers are visible on the CT image.

5. The system of claim 1, wherein the fiducial marker comprises an intersection of lines, wherein each line is a wire made of the radio-opaque material.

6. The system of claim 1, wherein fiducial marker comprises a plurality of points, wherein each point is a bead made of the radio-opaque material.

7. The system of claim 1, wherein the plurality of fiducial markers are arbitrarily arranged on the surface of the patient.

8. The system of claim 1, wherein the plurality of fiducial markers are arranged in proximity to an area of the patient that is subject to the medical procedure.

9. The system of claim 1, wherein the plurality of fiducial markers are arranged to cover an area of the patient that is subject to the medical procedure.

10. The system of claim 1, wherein the camera is a video camera, wherein the x-ray system is a fluoroscopy system.

11. The system of claim 1, wherein the camera is coupled to the instrument.

12. The system of claim 1, wherein the instrument is one of a guidewire, a biopsy needle, and a drill.

13. The system of claim 1, wherein the computer is further configured to calibrate an axis of the instrument to a line of sight of the camera.

14. The system of claim 1, wherein the spatial position of the instrument relative to the x-ray system is determined on the x-ray images during the medical procedure in real-time.

15. The system of claim 1, wherein the plurality of fiducial markers are positioned on the surface of the patient at the beginning the medical procedure.

16. The system of claim 1, wherein the computer is further configured to determine the spatial position relative to the camera for the subset of fiducial markers based on at least one camera calibration parameter.

17. The system of claim 1, wherein the computer is further configured to:
receive a magnetic resonance imaging (MRI) image acquired from an MRI system prior to the medical procedure
perform a registration of the x-ray image to the MRI image;
based on the registration of the MRI image to the x-ray image, display the spatial position of the instrument relative to the x-ray system as an overlay on the MRI image.

18. The system of claim 17, wherein none of the fiducial markers are visible on the MRI image.

19. A method for surgical navigation, comprising:
receiving an optical image acquired by a camera, said camera attached to an instrument configured for a medical procedure on a patient, wherein the instrument has a spatial position relative to the camera;
receiving an x-ray image acquired by an x-ray system configured to acquire x-ray images of the patient during the medical procedure;
for a plurality of fiducial markers positioned on the surface of the patient during the medical procedure and detectable by both the camera and the x-ray system, identifying a subset of the fiducial markers that are visible in the optical image and are also visible in the x-ray image, said fiducial markers comprising a radio-opaque material arranged as at least one of a line and a point;
determining, based on the optical image, a spatial position relative to the camera for each fiducial marker in the subset of fiducial markers;
determining, based on the x-ray image, a spatial position relative to the x-ray system for each fiducial marker in the subset of fiducial markers; and
determining, based on at least the spatial positions of the subset of fiducial markers relative to the camera and the spatial positions of the subset of fiducial markers relative to the x-ray system, a spatial position for the instrument relative to the x-ray system.

20. The method of claim 19 further comprising displaying the spatial position of the instrument relative to the x-ray system as an overlay on the x-ray images.

21. The method of claim 19 further comprising:
receiving a computed tomography (CT) image acquired from a CT system prior to the medical procedure;
performing a registration of the x-ray image to the CT image;
based on the registration of the CT image to the x-ray image, displaying the spatial position of the instrument relative to the x-ray system as an overlay on the CT image.

22. The method of claim 21, wherein none of the fiducial markers are visible on the CT image.

23. The method of claim 19, wherein the fiducial marker comprises an intersection of radio-opaque wires.

24. The method of claim 19, wherein the fiducial marker comprises a plurality of radio-opaque beads.

25. The method of claim 19, wherein the plurality of fiducial markers are arbitrarily arranged on the surface of the patient.

26. The method of claim 19, wherein the plurality of fiducial markers are arranged in proximity to an area of the patient that is subject to the medical procedure.

27. The method of claim 19, wherein the plurality of fiducial markers are arranged to cover an area of the patient that is subject to the medical procedure.

28. The method of claim 19, wherein the camera is a video camera, wherein the x-ray system is a fluoroscopy system.

29. The method of claim 19, wherein the camera is coupled to the instrument.

30. The method of claim 19, wherein the instrument is one of a guidewire, a biopsy needle, and a drill.

31. The method of claim 19 further comprising calibrating an axis of the instrument to a line of sight of the camera.

32. The method of claim 19, wherein the spatial position of the instrument relative to the x-ray system is determined on the x-ray images during the medical procedure in real-time.

33. The method of claim 19, wherein the plurality of fiducial markers are positioned on the surface of the patient at the beginning the medical procedure.

34. The method of claim 19, wherein determining the spatial position relative to the camera for the subset of fiducial markers is further based on at least one camera calibration parameter.

35. The method of claim 19 further comprising:
receiving a magnetic resonance imaging (MRI) image acquired from an MRI system prior to the medical procedure;
performing a registration of the x-ray image to the MRI image;
based on the registration of the MRI image to the x-ray image, displaying the spatial position of the instrument relative to the x-ray system as an overlay on the MRI image.

36. The method of claim 35, wherein none of the fiducial markers are visible on the MRI image.

37. A system for surgical navigation, comprising:
an instrument configured for a medical procedure on a patient;
a camera attached to the instrument, wherein the instrument has a spatial position relative to the camera;
an x-ray system configured to acquire x-ray images of the patient during the medical procedure;
a plurality of fiducial markers positioned on the surface of the patient dining the medical procedure, said fiducial markers being detectable by both the camera and the x-ray system; and
a computer configured to:
receive a two-dimensional (2D) optical image acquired by the camera;
receive a 2D x-ray image acquired by the x-ray system;
identify a subset of the fiducial markers that are visible in the optical image and are also visible in the x-ray image;
determine, based on the 2D optical image, a spatial position relative to the camera for each fiducial marker in the subset of fiducial markers;
determine, based on the 2D x-ray image, a spatial position relative to the x-ray system for each fiducial marker in the subset of fiducial markers; and
determine, based on at least the spatial positions of the subset of fiducial markers relative to the camera and the spatial positions of the subset of fiducial markers relative to the x-ray system, a spatial position for the instrument relative to the x-ray system.

38. The system of claim 37, wherein the computer is further configured to display the spatial position of the instrument relative to the x-ray system as an overlay on the 2D x-ray images.

39. The system of claim 37, wherein the computer is further configured to:
receive a 3D computed tomography (CT) image acquired from a CT system prior to the medical procedure;
perform a 3D-2D registration of the 3D CT image to the 2D x-ray image;
based on the 3D-2D registration of the 3D CT image to the 2D x-ray image, display the spatial position of the instrument relative to the x-ray system as an overlay on the 3D CT image.

40. The system of claim 39, wherein none of the fiducial markers are visible on the 3D CT image.

41. The system of claim 33, wherein the fiducial markers comprise a radio-opaque material arranged as at least one of a line and a point.

42. The system of claim 41, wherein the fiducial marker comprises an intersection of lines, wherein each line is a wire made of the radio-opaque material.

43. The system of claim 41, wherein fiducial marker comprises a plurality of points, wherein each point is a bead made of the radio-opaque material.

44. The system of claim 37, wherein the plurality of fiducial markers are arbitrarily arranged on the surface of the patient.

45. The system of claim 37, wherein the plurality of fiducial markers are arranged in proximity to an area of the patient that is subject to the medical procedure.

46. The system of claim 37, wherein the plurality of fiducial are arranged to cover an area of the patient that is subject to the medical procedure.

47. The system of claim 37, wherein the camera is a video camera, wherein the x-ray system is a fluoroscopy system.

48. The system of claim 37, wherein the camera is coupled to the instrument.

49. The system of claim 37, wherein the instrument is one of a guidewire, a biopsy needle, and a drill.

50. The system of claim 37, wherein the computer is further configured to calibrate an axis of the instrument to a line of sight of the camera.

51. The system of claim 37, wherein the 2D spatial position of the instrument relative to the x-ray system is determined on the x-ray images during the medical procedure in real-time.

52. The system of claim 37, wherein the plurality of fiducial markers are positioned on the surface of the patient at the beginning the medical procedure.

53. The system of claim 37, wherein the computer is further configured to determine the spatial position relative to the camera for the subset of fiducial markers based on at least one camera calibration parameter.

54. The system of claim 37, wherein the computer is further configured to:
receive a 3D magnetic resonance imaging (MRI) image acquired from an MRI system prior to the medical procedure;
perform a 3D-2D registration of the 3D MRI image to the 2D x-ray image;
based on the 3D-2D registration of the 3D MRI image to the 2D x-ray image, display the spatial position of the instrument relative to the x-ray system as an overlay on the 3D MRI image.

55. The system of claim 54, wherein none of the fiducial markers are visible on the 3D MRI image.

56. A method for surgical navigation, comprising:
receiving a two-dimensional (2D) optical image acquired by a camera, said camera. attached to an instrument configured for a medical procedure on a patient, wherein the instrument has a spatial position relative to the camera;
receiving a 2D x-ray image acquired by an x-ray system configured to acquire x-ray images of the patient during the medical procedure;
for a plurality of fiducial markers positioned on the surface of the patient during the medical procedure and detectable by both the camera and the x-ray system, identifying a subset of the fiducial markers that are visible in the optical image and are also visible in the x-ray image;
determining, based on the 2D optical image, a spatial position relative to the camera for each fiducial marker in the subset of fiducial markers;
determining, based on the 2D x-ray image, a spatial position relative to the x-ray system for each fiducial marker in the subset of fiducial markers; and
determining, based on at least the spatial positions of the subset of fiducial markers relative to the camera and the spatial positions of the subset of fiducial markers relative to the x-ray system, a spatial position for the instrument relative to the x-ray system.

57. The method of claim 56 further comprising displaying the spatial position of the instrument relative to the x-ray system as an overlay on the x-ray image.

58. The method of claim 56 further comprising:
receiving a 3D computed tomography (CT) image acquired from a CT system prior to the medical procedure;
performing a 3D-2D registration of the 3D CT image to the 2D x-ray image;
based on the 3D-2D registration of the 3D CT image to the 2D x-ray image, displaying the spatial position of the instrument relative to the x-ray system as an overlay on the 3D CT image.

59. The method of claim 58, wherein none of the fiducial markers are visible on the CT image.

60. The method of claim 56, wherein the fiducial markers comprise a radio-opaque material arranged as at least one of a line and a point.

61. The method of claim 60, wherein the fiducial marker comprises an intersection of lines, wherein each line is a wire made of the radio-opaque material.

62. The method of claim 60, wherein fiducial marker comprises a plurality of points, wherein each point is a bead made of the radio-opaque material.

63. The method of claim 56, wherein the plurality of fiducial markers are arbitrarily arranged on the surface of the patient.

64. The method of claim 56, wherein the plurality of fiducial markers are arranged in proximity to an area of the patient that is subject to the medical procedure.

65. The method of claim 56, wherein the plurality of fiducial markers are arranged to cover an area of the patient that is subject to the medical procedure.

66. The method of claim 56, wherein the camera is a video camera, wherein the x-ray system is a fluoroscopy system.

67. The method of claim 56, wherein the camera is coupled to the instrument.

68. The method of claim 56, wherein the instrument is one of a guidewire, a biopsy needle, and a drill.

69. The method of claim 56 further comprising calibrating an axis of the instrument to a line of sight of the camera.

70. The method of claim 56, wherein the spatial position of the instrument is determined on the x-ray images during the medical procedure in real-time.

71. The method of claim 56, wherein the plurality of fiducial markers are positioned on the surface of the patient at the beginning the medical procedure.

72. The method of claim 56, wherein determining the 3D spatial position for each of the fiducial markers visible in the optical image is further based on at least one camera calibration parameter.

73. The method of claim 56 further comprising:
receiving a 3D magnetic resonance imaging (MRI) image acquired from an MRI system prior to the medical procedure;
performing a 3D-2D registration of the 3D MRI image to the 2D x-ray image;
based on the 3D-2D registration of the 3D MRI image to the 2D x-ray image, displaying the spatial position of the instrument relative to the x-ray system as an overlay on the 3D MRI image.

74. The method of claim 73, wherein none of the fiducial markers are visible on the MRI image.

75. The system of claim 1, wherein the instrument is a drill guide.

76. The method of claim 19, wherein the instrument is a drill guide.

77. The system of claim 37, wherein the instrument is a drill guide.

78. The method of claim 56, wherein the instrument is a drill guide.
